(19) United States
(12) Patent Application Publication
(10) Pub. No.: US 2003/0198617 A1
(43) Pub. Date: Oct. 23, 2003

(54) Pharmaceutical tryptophan containing dipeptide compositions and methods of use thereof

(76) Inventors: Lawrence Green, Tacoma, WA (US); Nicolay V. Sinackevich, St. Petersburg (RU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/164,693

(22) Filed: Jun. 6, 2002

Related U.S. Application Data
(63) Continuation of application No. 09/557,102, filed on Apr. 21, 2000, now abandoned.
Application No. 09/557,102 is a continuation of application No. 08/522,232, filed on Feb. 26, 1996, now abandoned.
Application No. 08/522,232 is a 371 of international application No. PCT/US94/02354, filed on Mar. 4, 1994.
Application No. PCT/US94/02354 is a continuation-in-part of application No. 08/026,341, filed on Mar. 4, 1993, now abandoned.

(51) Int. Cl. A61K 38/20; A61K 38/19; A61K 38/18; A61K 38/05; A61K 31/704; A61K 31/496; A61K 31/497; A61K 31/4965; A61K 31/44

(52) U.S. Cl. 424/85.1; 424/85.2; 514/12; 514/19; 514/255.06; 514/252.13; 514/355; 514/37

(57) ABSTRACT

The present invention provides compositions and methods for treatment of a variety of disease states. The methods generally comprise administering to a host a therapeutically effective amount of a dipeptide having the formula X-Tryptophan or a pharmaceutically acceptable salt thereof, wherein x is glutamine, glutamate, leucine, or isoleucine. The present invention is useful for treatment of infections hyperimmune states, immunodeficiencies, and the like.

This application is a continuation-in-part of U.S. patent application No. (U.S. Ser. No.) 08/075,842, filed Jun. 10, 1993, and U.S. Ser. No. 07/783,518, filed Oct. 28, 1991, which are continuations of U.S. Ser. No. 07/678,129, filed Apr. 1, 1991, now abandoned; and a continuation-in-part of U.S. Ser. No. 08/026,341, filed Mar. 4, 1993, each of which is incorporated herein by reference.

### BACKGROUND OF THE INVENTION

The present invention relates generally to pharmaceutical compositions containing peptides having immunomodulating properties. More particularly, the present invention relates to pharmaceutical compositions of tryptophan-containing dipeptides and methods of use thereof.

The immune system performs critical functions in animals, including humans. These functions include, e.g., preventing and combating infection, surveillance and immunoablation of tumors, and the like. A loss of immune system activity often results in serious and life-threatening diseases. Such functional abnormalities may be present in any of the components of the immune system, e.g., granulocytes, lymphocytes, complement, etc. Animals having dysfunctional immune systems may be at increased risk for malignancies and infections.

Although functional abnormalities causing immunodeficiencies often have similar clinical presentations, the abnormalities may result from many etiologies. These causes may be, e.g., hereditary genetic abnormalities (e.g., Chediak-Higashi Syndrome, Severe Combined Immunodeficiency, Chronic Granulomatous Disease, DiGeorge Syndrome, and the like), toxin-related (e.g., radiation exposure, heavy metal poisoning, insecticide exposure, and the like), iatrogenic (e.g., chemotherapy-related neutropenia, glucocorticosteroid therapy, and the like), or infectious (e.g., HIV infection, other viral leukopenias, and tuberculosis). Patients with such diseases would benefit from methods for enhancing and stimulating the immune system.

Immunological stimulation, even in healthy individuals, may aid in the treatment of several diseases. Infectious diseases may be more effectively treated by stimulation of the immune system. The enhanced immunological response may work with other treatments to eliminate the infection more readily. Also, specific immune stimulation has been shown to reduce tumor size in some malignancies. Also, many drugs used for primary treatment of infections and malignancies have significant side effects. Therefore, it is desirable to reduce the dose of the primary drug whenever possible.

Diseases may also be caused by hyperactivity of the immune system. For example, collagen vascular diseases are associated with immunologically-mediated damage to the host tissue. Such diseases include multiple sclerosis, rheumatoid arthritis, Sjogren's syndrome, and the like. These diseases afflict many individuals and cause significant morbidity and mortality. Treatments generally include immune suppression. Unfortunately, generalized immune suppression often results in increased incidence of infections and malignancies as described above. Therefore, treatment of one disease places patients at risk for developing other, possibly life threatening, diseases.

Immune suppression is also used for enhancing graft compatibility following tissue transplantation. Graft-versus-host disease following bone marrow transplantation can cause particularly severe complications. The immune cells maturing from the engrafted bone marrow may recognize the host cells as “foreign.” The engrafted immune system may then attempt to reject the “foreign” tissue, i.e., the host's normal cells. Cytotoxic T-cells attack host cells and produce a clinical syndrome of multi-organ rejection. Therefore, immune suppression is necessary to prevent immunological ablation of host organs, such as liver, kidney, lungs, gastrointestinal tract, and the like.

Peptides derived from the thymus have been suggested as playing important immuno-regulatory roles in many animals, including humans. Some of these peptides have been demonstrated to induce saturation, differentiation, and function of T lymphocytes. For example, Thymosin fraction 5, a fraction of calf thymus extract, may restore immune function in athymic or immunodepressed individuals. Several peptides have been isolated from Thymosin fraction 5, including Thymosin-α₁ (28 amino acids, U.S. Pat. No. 4,079,127); Thymosin β₄ (44 amino acids, Low et al., Proc. Natl. Acad. Sci. USA, 78:1162-1166 (1981); Thymosin β₈ (39 amino acids, U.S. Pat. No. 4,389,343); and Thymosin β₉ (41 amino acids, U.S. Pat. No. 4,389,343). In some patients, these peptides may produce untoward side effects. WO92/17191 discloses L-Glu-L-Trp and pharmaceutical salts thereof for therapy of immunodepressed or hyperactive immune states and for the prevention and treatment of opportunistic infections in such states, EP-A-0346401 discloses H-L-Glu-L-Trp-OH for treating immune deficiency conditions such as following radiation exposure or as the result of chronic staphyloccal pyoderma infection, or following thymectomy. Uses of L-Glu-L-Trp for wound healing are also disclosed e.g. for promoting healing of burns, frostbile and corneal damage.

What is needed in the art are compositions and methods for modulating the immune system. Ideally, these compounds and methods would be able to stimulate suppressed or deficient immune systems as well as reduce immune hyperactivity. Also these compounds and methods should act to restore a natural balance to the immune system. Quite surprisingly, the present invention fulfills these and other related needs.

### SUMMARY OF THE INVENTION

The present invention provides pharmaceutical compositions comprising tryptophan-containing dipeptides. The dipeptides have the structure X-Trp. X may be any naturally-occurring amino acid, preferably a neutral polar or acidic amino acid. Generally X is glutamine, leucine, or isoleucine. The dipeptide is present in therapeutically effective amounts in the pharmaceutical compositions of the present invention with a pharmaceutically acceptable carrier.

Also provided are methods for treating a variety of disease conditions, such as, e.g., infections, hyperimmune states, immune deficient states, and the like. The infections may be viral, bacterial, mycobacterial, fungal, or parasitic. The methods generally comprise administering a therapeutically effective amount of a tryptophan-containing dipeptide having the formula X-Trp to a host suffering from a disease. In alternate embodiments, the methods of the present invention may further comprise administering additional therapeutic agents to the host. The host may be any animal, including humans.

The present invention provides pharmaceutical compositions and methods useful for regulation of a host's immune system. The immune system can be regulated to become more active or less active, depending on the level of immune competence at the time of administration. It is believed that the tryptophan-containing dipeptides of the present invention are active as signal peptides in an immunoregulatory feedback system similar to that suggested in Birr, Thymic Hormones and Lymphokines, Plenum, A. Goldstein ed. 97-107 (1984). It is believed that such peptides are active components of an endogenous immunoregulatory system for maintaining a balance within the immune system of humans and other animals. The methods and compositions of the present invention can, therefore, be used to stimulate immune responses in, e.g., immunodeficient hosts, infected immunocompetent hosts, hosts having tumors, for augmentation of vaccines, and the like; and also to suppress hyperimmune states, such as, e.g., rheumatoid arthritis, systemic lupus erythematosus, Sjögren's syndrome, graft rejection, allergic conditions, graft-versus-host disease, and the like.

As used herein, the terms “immunomodulator” and “immunomodulating” encompass the activity of restoring the natural balance to a host's immune system. This includes enhancing or restoring the subject's immune system, as evidenced by measurable blood parameters and/or the patient's improved ability to combat infection or disease, and the ability to heal tissue. Hence, immunomodulation encompasses improvement of the immune system due to an immunodeficient state (for example, caused by removal of the thymus), and/or an immunodepressed state (for example, caused by exposure to radiation). Furthermore, the present invention provides for modulation of the immune system by lowering blood parameters and other indicia of the immune state if these indicia are abnormally elevated. The present invention encompasses the therapeutic method of treating the immunodeficient, immunodepressed or elevated immune state per se, thus providing prophylaxis against infection and disease, as well as a treatment of infection, disease or wound by enhancing the immune system.

### DESCRIPTION OF THE PREFERRED EMBODIMENT

The pharmaceutical compositions of the present invention comprise a therapeutically effective amount of a dipeptide having the formula X-Tryptophan or a pharmaceutically acceptable salt thereof, wherein X is any naturally-occurring amino acid; and a pharmaceutically acceptable carrier. Generally, X will be glutamine, leucine, or isoleucine.

The compositions of the present invention may also contain cyclic and polymeric forms of tryptophan-containing dipeptides as described above. Up to three dipeptide subunits may be joined by peptide bonds to form the polymeric peptide forms. such forms may have the following formulas:

X-Trp-Y-Trp,

or

X-Trp-Y-Trp-Z-Trp,

or pharmaceutically acceptable salts thereof, wherein X, Y, and Z may be any naturally-occurring amino acids. X, Y, and Z may be the sane or different amino acids. Generally, at least one of X, Y, and Z will be glutamine, leucine, or isoleucine.

The dipeptides and dipeptide polymeric forms in the pharmaceutical compositions ay also be cyclic. The cyclic forms may have 1, 2, or 3 dipeptide subunits and have the general formula

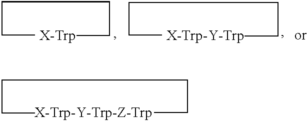

or pharmaceutically acceptable salts thereof, wherein X, Y, and Z may be any naturally-occurring amino acids. X, Y, and Z may be the same or different amino acids. Generally, at least one of X, Y, and Z will be glutamine, leucine, or isoleucine.

It will be further appreciated that simple derivatives of any of the aforementioned peptides which do not significantly alter the activity of such peptides fall within the scope of the present invention and are envisioned by the inventors. Such derivatized peptides include acylated derivatives, amidated derivatives, and the like. A screening assay for identifying a candidate antimicrobial drug or tryptophan-containing dipeptide in accordance with the present invention, comprises the steps of: (a) synthesizing a derivative of a dipeptide having the formula X-Trp; (b) introducing the derivative into a T-cell rosette assay as a test article; and (c) determining that the zest, article has substantially the same activity as the dipeptide in the T-cell rosette assay. The present invention also envisions easily hydrolyzed compounds which release tryptophan-containing peptides into body tissues and fluids.

The tryptophan-containing dipeptides in the compositions of the present invention are members of a class of small signal peptides (6 amino acids or less) that regulate receptor-ligand affinity. This class of signal peptides, hereinafter referred to as Cytomedines, participate in restoration and maintenance of normal cellular physiology and morphogenesis in a homeostatic manner. Disease-causing insults, such as genetic abnormalities, environmental insults, and the like, inhibit normal regulatory processes by preventing synthesis of Cytomedines. Any factor causing a disruption of the normal dynamic cellular state regulated by Cytomedines causes progressive alteration of certain cellular events that may present as a clinical illness. The loss of regulatory function way result in several pathological conditions.

Administration of cytomedines, such as in the compositions and methods of the present invention, can restore normal regulators states to the affected cells. Furthermore, normalization of cellular regulation can restore normal physiological function. By restoring normal physiological function, it is believed that cytomedines can reverse disease processes and effectively treat a variety of conditions.

Cytomedines are believed to interact with cellular receptors. Traditionally, cellular receptors have been believed to bind specific ligands with a specific affinity. Binding (or non-binding) of the ligand to the cellular receptor is believed to induce certain cellular functions. It is further believed that cytomedines interact with these cellular receptors. In addition to the ligand-binding site, however, the receptors apparently have separate cytomedine-binding sites. It is believed that receptor-cytomedine binding can alter the conformation of the ligand-binding site so as to increase the affinity of the receptor for the ligand, thereby altering the response of the cell to a particular concentration of ligand within the microenvironment of the cell. Different cytomedines and different cytomedine concentrations can have varying effects on the binding affinity between the receptor and the ligand.

For example, when the dipeptide Glu-Trp interacts with T cells, an increase of intracellular cAMP concentrations is observed in the cells. This in turn activates the intracellular protein kinase activity that is important in the immunological function of many cells, including T cells.

Based upon current knowledge, it is believed that the tryptophan-containing signal peptides of the present invention reversibly associate with specific cellular receptors, namely “CD2” receptors, thereby inducing conformational changes in the receptor which “trigger” intracellular mechanisms resulting in up regulation of adenylate cyclase and an increase in AMP, while simultaneously increasing the affinity of the CD2 receptor for its “target” ligand. This increase in affinity is believed to heighten the interaction between these cells and their natural ligands, thereby facilitating such interaction and encouraging cellular responses to such interaction.

The pharmaceutical compositions of the present invention may be employed in pharmaceutical preparations for a variety of therapeutic uses. The preparations may be administered to a variety of hosts for therapeutic purposes. Suitable hosts include human and non-human primates, domestic animals including dogs, cats, rodents, birds, horses, cows, pigs, fish, and the like.

The compositions of the present invention may also find use for pre- or post-exposure prophylaxis, e.g., human immunodeficiency virus or hepatitis virus prophylaxis following “dirty needle” injuries to health care workers or routinely accompanying blood transfusions or to persons in danger of becoming exposed to infected body or culture fluids, and the like. The peptides of the present invention are particularly useful for augmentation of vaccinations. By “augmentation of vaccines”, it is meant that the level and/or duration of complete or partial protection from disease obtained from vaccination is enhanced.

Administration of the compositions of the present invention in conjunction with a vaccine will enhance the immune response to the vaccine providing both a higher level of immunity and a prolonged anamnestic response. The compositions can be administered prior to, simultaneously with, or following vaccination. Generally, the compositions will be administered prior to, or simultaneously with, vaccination.

The pharmaceutical compositions of the present invention are intended for parenteral, topical, oral, intranasal, or local administration for prophylactic and/or therapeutic treatment. Preferably, the compositions of the present invention are administered intramuscularly or intranasally. As the compositions of the present invention may be administered parenterally, i.e., intravenously, subcutaneously, intramuscularly, or intrathecally, the present invention provides pharmaceutical preparations for parenteral administration which comprise a solution of a tryptophan-containing dipeptide, or polymeric or cyclic form thereof, dissolved in a pharmaceutically acceptable carrier, preferably an aqueous carrier. A variety of aqueous carriers may be used, e.g., water, buffered water, 0.4% saline, 0.3% glycine, and the like, including proteins and/or glycoproteins for enhanced stability, such as albumin, lipoprotein, globulin, and the like. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The active peptides of the pharmaceutical preparations according to the present invention may be used as free peptides or in the form of a water soluble pharmaceutically acceptable salt, such as a sodium, potassium, ammonium or zinc salt.

In addition to the peptides and physiologically acceptable carriers, the pharmaceutical preparations may include other active ingredients which independently impart an activity to the composition. Anti-infective agents, such as antibacterial agents, anti-fungal agents, anti-viral agents, and anti-parasitic agents are particularly suitable for addition to the pharmaceutical compositions of the present invention for use of the compositions to treat infectious diseases. Other bioactive compounds may also be added to the present compositions. Such compositions include, e.g., oncolytic agents, an interferon, an interleukin, tumor necrosis factor, a transforming growth factor, leukemia inhibitory-factor, a colony stimulating factor, anesthetics, and the like.

The concentration of the peptides in these pharmaceutical compositions can vary widely, i.e., from about 0.001% to as much as 15 or 20% by weight and will be selected primarily by fluid volumes, viscosities, etc., in accordance with the particular mode of administration selected. When utilized intramuscularly as an injection solution with the active ingredient in a therapeutically effective immunopotentiating amount of about 0.001 to 0.01% by weight. If prepared in the form of a tablet, capsule or suppository, it is preferred that the active ingredient be present in an amount of about 0.1 mg per tablet, suppository or capsule. In such form, the capsule, suppository or tablet may also contain other conventional excipients and vehicles such as fillers, starch, glucose, etc. In topical preparations, the peptides are generally contained in urea-based emollients, petroleum-based ointments, and the like at concentrations of about 0.1 to 10,000 parts per million, preferably about 1 to 1000 parts per million, and most preferably about 10 to 100 parts per million. Actual methods for preparing parenterally, orally, and topically administrable compounds will be known or apparent to those skilled in the art and are described in detail in, for example, Remington's Pharmaceutical Science, 17th ed., Mack Publishing Company, Easton, Pa. (1985), which is incorporated herein by reference.

The peptides in the present compositions induce changes at the cellular level, resulting in subsequent independent cellular processes which no longer relate to the fate of the peptide. So it is observed in many instances that the effects of the peptide are long lasting weeks and months later, despite the rapid degradation of the peptide within minutes or hours.

These simple peptides comprised of naturally occurring amino acids are known to be rapidly degraded by a variety of endo- and exo-peptidases, some of which reside on specific surface membranes, and others of which are found throughout the body and circulation. It is well understood that such peptides, degraded to the constituent amino acids, the basic building material of all proteins, are seldom if ever directly excreted through the urine or feces. The constituent amino acids derived from such peptides are subsequently incorporated into new proteins, or metabolized, derivatized, or excreted as is the fate of the naturally occurring amino acids which are ingested or derived from naturally occurring host peptides.

The peptides in the present compositions are typically biologically active at a dose of about 0.5 to about 10, preferably about 1 to about 5 mcg/kg. It will be appreciated that healthcare professionals can conduct escalated dose studies to determine precise dosages for specific patients. A frequently observed pattern with Cytomedine administration is the same approximate peak profile for activity of different therapeutic peptides. These peak profiles are observed both in vivo, and in vitro and appear closely similar across species for a wide number of measured parameters. These observations in conjunction with known cytomedine-membrane receptor binding (e.g., to CD2) is consistent with the peptides' proposed mechanism of action through causing conformational changes in the receptor that alters the affinity of binding of the receptor to its target ligand. Further, cytomedine binding to receptors is apparently a reversible equilibrated process which may be subsequently changed by introduction of other active peptides. The binding site appears either directly or indirectly under the influence of competitor peptides. Thus, the cytomedine's primary role appears as a “regulator” of the receptor, inducing conformation changes altering affinity interactions, and under certain conditions inducing intracellular secondary messenger activity.

This knowledge provides a method for calculating therapeutically effective doses of peptides in the present compositions. The number of CD2 receptors on the normal T-cell is known to occupy approximately 1% of the cell surface, and is estimated in the range of 10⁴ to 10⁵ receptors per cell. Therefore. it is possible to calculate, given the number of T-cells per ml determined in a rosette experiment as is well known in the art, the number of CD2 receptors per ml. Rosetting may be performed as described in Kontny et al., Immunology, 77:196-200 (1992), incorporated herein by reference. Cytomedine resetting in both trypsinized and nontrypsinized T-cells reveal that the concentration of cytomedine required to observe peak activity is proportional to the number of CD2 receptors per ml. Peak cytomedine activity generally occurs around 1 to 10 mcg/kg. As these peptides have very large volumes of distribution and are freely disseminated throughout the tissues, their concentrations can be approximated as 1 to 10 mcg/liter (1 kg=1 liter). Using the known molecular weight for each peptide and Avogadro's number, the number of cytomedine molecules per ml can be then calculated, and consistently reveals the ratio of Receptor Molecules/ml can be then calculated, and consistently reveals the ratio of Receptor Molecules/ml to Cytomedine Molecules/ml to be 1:1 to 1:1000.

$$1 : 1000 \frac{Receptors \text{/} ml}{Cytomedines \text{/} ml} = 1 … to … 1000 \frac{Receptors \text{/} ml}{Cytomedines \text{/} ml} = 1 : 1 to$$

The above equation provides a means to calculate the maximally effective dose of a particular cytomedine provided the molecular weight is known (from the sequence of amino acids), and the target receptor number per cell (targeted by the Cytomedine) are known. It is possible to predict the dose response range for efficacy with the association of receptor to Cytomedine ranging from 1 to 1000 per receptor site.

For example, the peak effect for Glu-Trp (IM862) rosette activity occurs at approximately 0.0006 mcg/ml. Considering the molecular weight for IM862 (˜300 Daltons), and Avogadro's number 6×10²³,

Number of IM862 molecules/ml=1.2×10¹²

The number of CD2 per ml can be calculated as it is known that there are approximately 10⁵ CD2 per cells, and 10⁶ cells per ml in the rosette experiment,

Number of CD2 receptors/ml=1×10¹¹

The ratio of receptors to IM862 is 1:10 in this example. The number of receptors per cell is estimated based on a number of experiments using monoclonal antibodies, and probably varies from species to species. Nevertheless, this example serves to guide one in predicting the range of concentrations to be tested in developing therapeutically useful cytomedines. It is believed that the range in ratios could be reduced if the number of receptors was known with more precision, and it is probably, considering the, association constant between receptor and cytomedine that the optimal ratio is 1:1 to induce a conformational change.

Therefore, calculation of the dose range of the cytomedine, including the tryptophan-containing dipeptides described herein, for any species may be accomplished by

1. Determining the target receptor.

2. Determining the number of receptors per cell and the number of cells per ml (or gram of tissue).

3. Calculating the number of receptors per ml (receptors/cell×# cells/ml), to determine the dose “D”. Assume volumes translate directly to weights, which is based on the principle of large volumes of distribution for small peptides.

4. The peak activity for the cytomedine for the specified receptor will be within the range “D to 1000D”.

5. To convert this number of mcg/ml or mcg/kg, divide “D” by Avogadro's number, multiply by the peptide (cytomedine) Dalton weight in units, and express this number in mcg/ml or mcg/kg as is appropriate.

Alternatively, determination of an effective amount of peptide to treat hosts afflicted with different ailments may be determined through standard empirical methods which are well known in the art. For example, immunomodulation may be monitored by serial determinations of leukocyte count, sheep red blood cell erythrocyting activity, determination of relative and absolute levels of different leukocyte subsets (e.g., CD4 and CD8 subsets of T lymphocytes), sedimentation rates, C-reactive protein levels, immunoglobulin levels (particularly those directed at self-antigens), complement levels, and like, as well as general organ function of the host. Atopic states may be evaluated by challenges to allergens and determination of IgE levels. Leukocytic disorders may be monitored by determination of white blood cell counts and leukocyte function assays. Vaccine augmentation may be monitored by repeated challenge of antigen, either virulent or attenuated, and observation of the host's immune response to the challenge.

Compositions of the invention are administered to a host already suffering from an infection, as described above, in an amount sufficient to cure or at least partially arrest the disease and its complications. An amount adequate to accomplish this is defined as a “therapeutically effective dose.” Amounts effective for this use will depend on the severity of the infection or disease and the weight and general state of the patient being treated, but generally range from about 0.1 μg/kg to about 5000 μg/kg host body weight of peptide per day, more commonly about 0.2 μg/kg to about 1000 μg/kg host body weight of peptide per day, usually about 0.5 μg/kg to about 100 μg/kg host body per day, more usually about 0.75 μg/kg to about 20 μg/kg host body weight per day, and preferably about 1 μg/kg to about 5 μg/kg host body weight per day. Maintenance dosages over a prolonged period of time may be adjusted as necessary. Typical total daily doses are about 50 to 100 μg in adults, about 50 μg in children 7-14 years of age, about 20-30 μg in children 4 to 6 years of age, about 10-20 μg in children 1-3 years of age and about 10 μg in children less than one year of age. The compositions may be administered once daily or more often as desired. Treatment of acute conditions generally will occur over about 3-10 days. Treatment of chronic conditions or prophylactic treatments have the same course, but can be repeated after as long as about 1-6 months or longer. In some instances, it may be desirable to administer the compositions intermittently on a daily basis for periods of about 2 to about 20 days, preferably about 3 to about 14 days, more preferably about 4 to about 10 days which are repeated at least about 15 days, preferably about 20 days or as much as about 1 to 6 months or more.

It must be kept in mind that the materials of the present invention may be employed in serious disease states, that is, life-threatening or potentially life threatening situations. In such cases, in view of the minimization of extraneous substances and general lack of immunogenicity when a human-derived polypeptide is employed to treat human hosts, it is possible and may be felt desirable by the treating physician to administer substantial excesses of these compositions. This is not generally believed to be effective, however. For veterinary uses higher levels may be administered as necessary while avoiding, however, undesirable toxicities.

In prophylactic applications, compositions of the present invention are administered to a patient susceptible to or otherwise at risk for infection, anemia, or other disorder that may be treated by the methods of the present invention. Such an amount is defined to be a “prophylactically effective dose.” In this use, the precise amounts again depend on the patient's state of health and weight, but are generally in the ranges described above for therapeutic use. Prophylactic administration may be particularly desirable for hosts that have been exposed or at risk for exposure of infectious diseases, e.g., health-care workers, travellers, family members of infected individuals, immunosuppressed persons, and the like. The compositions of the present invention can be used for prophylaxis against common illnesses such as rhinoviruses, orthomyxoviruses, adenoviruses, α-hemolytic Streptococcus, and the like. The compositions of the present invention can be administered for surgical prophylaxis to lessen the risk of infectious complications. The compositions can also be used to inhibit organ rejection. Such organs can include skin, heart, lung, kidney, bone, liver, pancreas, tendon, and the like. The present compositions are particularly useful when used prophylactically to inhibit rejection of skin grafts.

Single or multiple administrations of the compositions can be carried out with the dose levels and pattern being selected by the treating physician or veterinarian. In any event, the pharmaceutical preparations should provide a quantity sufficient to effectively treat, prevent, or inhibit disease in the host.

For the treatment of infection, the pharmaceutical preparations of the present invention may be administered alone or as adjunct therapy. The compositions may be administered with, e.g., antibiotics, anti-viral compounds, anti-fungal compounds, and anti-parasitic compounds. When employed to enhance a host's immune response to a tumor through immunomodulation, the peptides of the present invention may be administered with a variety of compounds for the treatment of malignancy, graft-versus-host disease, hyperimmune states, and the like. When administered as adjunct therapy, the compositions of the present invention may be administered in conjunction with the other treatment modalities, or separately at different intervals.

The tryptophan-containing peptides in the compositions of the present invention may be synthesized by a variety of techniques well known in the art. Generally, the peptides will be prepared in solution or on a solid support by conventional peptide synthesis, including the Merrifield solid state peptide synthesis technique. For example, an amino and side chain protected derivative of an activated ester of Glx is reacted with side-group protected Trp, attached to the solid phase at its C-terminus. After elimination of the alpha-amino protecting group, the peptide maybe cleaved from the solid phase or another amino acid added in a similar fashion. Additional amino acids are serially added. The peptides are cleaved by highly acidic cleavage that also typically removes protecting groups. The peptides may then be isolated and lyophilized and stored for future use. Suitable techniques of peptide synthesis are described in detail in Stewart and Young, Solid Phase Peptide Synthesis, 2d edition, Pierce Chemical Company, 1984; and Tam et al., J. Am. Chem. Soc., 105:6442 (1983), both of which are incorporated herein by reference.

Alternatively, hybrid DNA technology may be employed for expression of the desired peptide in transformed eukaryotic or prokaryotic host cells. See, for example, Maniatis et al., Molecular Cloning, A Laboratory Manual, Cold Spring Harbor Laboratory, 1982, incorporated herein by reference.

The present invention also provides methods for treating a variety of disease states in a host. As noted above, the host may be any of a variety of animals, including humans, non-human primates, dogs, cats, horses, birds and fowl, cattle, fish, swine, and the like. The methods generally comprise administering to the host a therapeutically effective amount of a dipeptide having the formula X-Tryptophan or a pharmaceutically acceptable salt thereof, wherein X is a naturally-occurring amino acid. Generally, the amino acid will be glutamine, glutamate, leucine, or isoleucine. Also included are methods for treating disease states in a host administering to the host a therapeutically effective amount of a polymer or cyclic form of a dipeptide having the formula X-Tryptophan or a pharmaceutically acceptable salt thereof, wherein X is a naturally-occurring amino acid. The polymer and cyclic dipeptide forms may have as many as 3 tryptophan-containing dipeptide subunits with the following formulas:

X-Trp-Y-Trp,

X-Trp-Y-Trp-Z-Trp,

where X, Y, and Z are naturally-occurring amino acids. Similar to the linear dipeptide forms, X, Y, and Z will generally be glutamine, glutamate, leucine, or isoleucine. X, Y, and Z may be the same or different amino acids. The dose of the peptides is generally about 1 to 10 μg/kg of host body weight.

In some instances, the peptide compositions may be administered with other agents for the treatment of the disease state. Often, the dose of the additional agents may be less than standard dosages.

Pharmaceuticals that may be administered in conjunction with the compositions of the present invention include, e.g., anti-infectives such as Penicillin G. Penicillin V, Methicillin, Nafcillin, Oxacillin, Cloxacillin, Dicloxacillion, Ampicillin, Amoxicillin, Bacampicillin, Cyclacillin, Carbenicillin Indanyl, Ticarcillin, Mezlocillin, Piperacillin, Cephalothin, Cefazolin, Cephapirin, Cephradine, Cephalexin, Cefadroxil, Cefamandole Nafate, Cefuroxime, Cefonicid, Ceforanide, Cefaclor, Cefoxitin, Cefotetan, Cefmetazole, Cefataxime, Ceftizoxime, Ceftriaxone, Ceftazidime, Cefoperazone, Moxalactam, Cefixime, Erythromycin, Stearate, Ethylsuccinate, Estolate, Lactobionate, Gluceptate, Azithromycin, Clarithromycin Oxytetracycline, Demeclocycline, Doxycycline, Minocycline, Amikacin Sulfate, Gentamicin Sulfate, Intrathecal, Kanamycin Sulfate, Netilmicin Sulfate, Streptomycin Sulfate, Tobramycin Sulfate, Neomycin Sulfate, Sulfadiazine, Sulfamethizole, Sulfisoxazole, Sulfisoxazole Acetyl, Sulfamethoxazole, Trisulfapyrimidines, Phenazopyridine, Erythromycin Ethylsuccinate, Trimethoprim, Ciprofloxacin, Ciprofloxacin Hydrochloride, Enoxacin, Lomefloxacin Hydrochloride, Norfloxacin, Ofloxacin, Vancomycin Hydrochloride, Teicoplanin, Rifampin, Metronidazole, Metronidazole Hydrochloride, Polmyxins, Bacitracin, Methenamine, Methenamine Hippurate, Methenamine Mandelate, Nitrofurantoin, Phenazopyridine Hydrochloride, Silver Nitrate, Acetic Acid, Domeboro Solution, m-Cresyl Acetate, Coly-Mycin S Otic, Cortisporin, Tridesilon, Ciclopirox olamine, Clioquinol, Griseofulvin, Fulvicin, Grisactin, Grisactin Ultra, Grifulvin V, Halaprogin, Pyrithione zinc, Selenium sulfide, Tolnaftate, Undecylenic Acid, Naftfine, Terbinafind, Imidazole, Econazole, Ketoconazole, Miconaxole nitrate, Monistat-Derm, Oxiconazole nitrate, Sulconazole nitrate, Bis-triazoles, Intraconazole, Amphotericin B, Nystatin, Mycolstatin, Nilstat, Butoconazole, Clotrimazole, Ketoconazole, Miconazole nitrate, Tioconazold, Fluconazole, Intraconazole, Terconazole, Nystatin, Mycostatin, Nilstat, O-V Statin, Cantharidin, Interferon Alfa-2b, Interferon Alfa-n3, Intralesional, Podophyllin Resin, Podofilox, Salicylic Acid, Benzylbenzoate, Crotamiton, Lindane, Malathion, Permethrin, Phrethrins, Piperonyl Butoxide, Sulfur, Isoniazid, Pyrazinamide, Ethambutol, Capreomycin Sulfate, Cycloserine, Ethambutol Hydrochloride, Ethionamide, Clofazimine, Dapsone, Ethionamide, Itraconazole, Potassium Iodide Flucytosine, Chloroquine phosphate, Hydroxychloroquine phosphate, Chloroquine hydrochloride, Quinine sulfate, Pyrimethamine/sulfadoxine, Mefloquine, Quinidine gluconate, Dilozanide Furoate, Eflornithine Hydrochloride, Furazolidone, Iodoauinol, Melarsoprol, Metronidazole, Nifurtimox, Paramomycin Sulfate, Pentamidine Isethionate, Primaquine Phosphate, Quinine Sulfate, Sodium Stibogluconate, Meglumine Antimoniate, Trimetrexate Glucuronate, Pyrimethamine, Albendazole, Diethyclcarbamazine Citrate, Ivermectin, Mebendazole, Metrifonate, Niclosamide, Oxamniquine, Pyrantel Pamoate, Suramin Sodium, Thiabendazole, Cytarabine, Idoxuridine, Trifluridine, Vidarabine, Acyclovir, Zidovudine, Ribavirin, Bromovinyldeoxyuridine, Fluoroiodoaracytosine, Amantadine, Acemannan, Amphotericin B methyl, Ampligen, Castanospermine, Soluble CD₄, Dextran sulfate, Dideoxycytidine, Dideoxyinosine, Didihydrodideoxythymidine, Foscarnet sodium, Fusidic acid, HPA-23, Isoprinosine, Penicillamine, Peptide T, Ribavirin, Rifabutin, Zidovudine, Interferon Alfa-2b, Didanosine, Foscarnet Sodium, Zalcitabine, and the like.

Other adjunct treatments may include, e.g., anti-inflammatories such as Salicylates, Diclofenac Sodium, Etodolac, Fenoprofen Calcium, Flurbiprofen, Ibuprogen, Ketoprofen, Meclofenamate Sodium Monohydrate, Nabumetone, Naproxen, Napproxen Sodium, Oxaprozin, Phenylbutazone, Piroxicam, Sulindac, Tolmetin Sodium, Hydroxychloroquine Sulfate, Methotrexate, Penicillamine, Sulfasalazine, Aurothioglucose, Gold Sodium Thiomalate, Auranofin, Adrenal Corticosteroids, Azathioprine, Colchicine, Corticotropin, Fenoprofen Calcium, Allopurinol, Probenecid, Sulfinpyrazone, Probenecid, Colchicine, and the like; antihistamines such as e.g., Amino Alkylethers, Clemastine Fumarate, Tripelennamine Citrate, Tripelennamine Hydrochloride, Pyrilamine Maleate, Chlorpheniramine Maleate, Brompheniramine Maleate, Dexchlorpheniramine Maleate, Triprolidine Hydrochloride, Methdilazine, Methdilazine Hydrochloride, Promethazine Hydrochloride, Trimeprazine Tartrate, Azatadine Maleate, Cyproheptadine Hydrochloride, Hydroxyzine Hydrochloride, Hydroxyzine Pamoate, Acrivastine, Astemizole, Cetirizine Hydrochloride, Levocabastine Hydrochloride, Loratadine, Terfenadine, Ethanolamines, Ethylenediamine, Alkylamines, Phenothiazine, and the like; immunomodulators such as, e.g., Glucocorticoids, Acetate, Cypionate, Sodium Phosphate, Sodium Succinate, Acetate, Tebutate, Azathioprine, Azathioprine Sodium, Chlorambucil, Cyclophosphamide, Methotrexate, Methotrexate Sodium, Cyclosporine, Muromonab-CD3, Aldesleukin, BCG vaccine, Interferon Gamma-1b, Levamisole, Pegademase Bovine, Sargramostin, Filgrastim, Immune Glbulin, Lymphocyte Immune Clobulin, Muramyl Dipeptide, Thymic Hormones; vaccines such as Viral Vaccines, Toxoids, Meningococcal Polysaccharide vaccine, Diphtheria Antitoxin, Tetanus, Prophylaxis, Tetanus Immune Clobulin, Pertussis Vaccine, Measles Vaccine, Mumps Vaccine, Rubella Vaccine, PRP-D, Polysaccharide, PRP-OMP, Rabies Immune Globulin, BCG Vaccine, Cholera Vaccine, Meningococcal Polysaccharide Vaccine, Plague Vaccine, Smallpox vaccine, Vaccine Immune Globulin, Typhoid Vaccine, Yellow Fever Vaccine, Varicella-Zoster Immune Globulin, Botulism Antitoxin Trivalent, Cytomegalovirus Immune Globulin; oncolytics such as, e.g., Chlorambucil, Cyclophosphamide, Ifosfamide, Mechlorethamine Hydrochloride, Melphalan, Thiotepa, Busulfan, Procarbazine Hydrochloride, Carmustine, Lomustine, Streptozocin, Cisplatin, Carboplatin, Dacarbazine, Altretamine, Mesna, Methotrexate, Leucovorin Calcium, Cytarabine, Floxuridine, Fluorouracil, Cladribine, Fludarabine, Mercaptopurine, Pentostatin, Thioguanine, Hydroxyurea, Bleomycin Sulfate, Dactinomycin, Daunorubicin Hydrochloride, Doxorubicin Hydrochloride, Idarubicin Hydrochloride, Mitomycin, Mitoxantrone Hydrochloride, Plicamycin, Vinblastine Sulfate, Vincristine Sulfate, Etoposide, Paclitaxe, Teniposide, Asparaginase, Prednisone, Prednisolone, Dexamethasone, Methylprednisolone, Diethylstilbestrol, Chlorotrianisene, conjugated estrogen, Esterified estrogens, Estone, Ethinyl Estradiol, Estramustine Phosphate Sodium, Tamoxifen Citrate, Fluoxymesterone, Methyltestosterone, Testolactone, Testosterone Propinate, Flutamide, Goserelin Acetate, Leuprolide Acetate, Hydroxyprogesterone Caproate, Medroxyprogesterone Acetate, Megestrol Acetate, Aminoglutethimide, Mitotane, Aldesleukin, Interferon Alfa-2a, BCG, Isotretinoin, Levamisole, Octreotide Acetate, Cyclophosphamide, Ifosfamide, Mechlorethamine Hydrochloride, Melphalan, Mesna, Busulfan, Carmustine, Lomustine, Nimustine, Semustine, Streptozocin, Cisplatin, Carboplatin, Iproplatin, Procarbazine Hydrochloride, Dacarbazine, Altretamine, Sodium Phosphate P 32, Chromic Phosphate P 32, Methotrexate, Methotresate Sodium, Methotrexate, Trimetrexate, Fluorouracil, Floxuridine, Azacitidine, Tegafur, Cladribine, Fludarabine Phosphate, Mercaptopurine, Pentostatin, Thioguanine, Tiazofurin, Hydrocyurea, Caracemide, Buthionine Sulfoximine, Eflornithine Hydrochloride, Mitoguazone, Phosphonoacetyl, Brequinar Sodium, Doxorubicin Hydrochloride, Idarubicin Hydrochloride, Epirubicin Hydrochloride, Menogaril, Razoxane, Bleomycin Sulfate, Dactinomycin, Mitomycin, Plicamycin, Didemnin B, Echinomycin, Deoxyspergualin, Mitoxantrone Hydrochloride, Amsacrine, Amonafide, Merbarone, Piroxantrone Hydrochloride, Vinblastine Sulfate, Vincristine Sulfate, Vindesine Sulfate, Etoposide, Teniposide, Paclitaxel, Homoharringtonine, Asparaginase, Mitotane, Estramustine Phosphate Sodium, Tamoxifen Citrate, Leuprolide Acetate, Goserelin Acetate, Buserelin Acetate, Aminoglutethimide, Interferon Alfa-2a, Interferon Alfa-2b, Interferon Beta, Interleukin 2, Tumor Necrosis Factor, BCG Live. BCG Vaccine, Monoclonal Antibodies, Flavone Acetic Acid, Hexamethylene-Bis-Acetamide, Isotretinoin, Levamisole Hydrochloride, N-Methyformamide, Octreotide Acetate, and the like.

A variety of disease states may be treated by the methods of the present invention. Infectious diseases may be treated. The infections may be bacterial, viral, fungal, or parasitic. The methods may be practiced in immunocompromised or immunocompetent hosts. Localized or disseminated infections may be treated by the present methods. The infections may be in any organ, e.g., lungs, bone, kidney, central nervous system, heart, skin and soft tissues (e.g., post-traumatic infections), reproductive organs (orchitis, pelvic inflammatory diseases, and the like), liver and the like.

Infectious diseases may be treated by the methods of the present invention. Infections with a variety of prokaryotes may be treated. For example, gram positive bacteria (e.g., Staphylococcus, Streptococcus, Actinomyces, and the like), gram negative bacteria (e.g., Enterobacteriaceae, Bacillus, and the like) infections may be treated by the present methods. Often, anti-infective agents also may be administered to the host. For example, when treating bacterial infections an antibiotic, such as a penicillin, cephalosporin, aminoglycoside, macrolide, sulfa, fluoroquinolone, or tetracycline, may be used as adjuvant therapy. This provides an additional mechanism for clearing the infection from the host.

The methods of the present invention may be practiced for the treatment of infection by mycobacterial organisms, such as Mycobacterium tuberculosis, Mycobacterium intracellulare, Mycobacterium leprae, Mycobacterium avium, Mycobacterium bovis, Mycobacterium kansasii, Mycobacterium paratuberculosis, and the like. The infection may be localized or generalized, e.g., pulmonary and disseminated lesions of Mycobacterium tuberculosis.

The methods for treating these diseases may further comprise administering at least one anti-infective agent to the host. The anti-infective agent will generally be administered according to its standard dosage schedule. For example, treatment of Mycobacterium tuberculosis infections may comprise administering the dipeptides (or corresponding polymeric or cyclic forms) to the host in conjunction with standard therapy, such as isoniazid, rifampin, ethambutol, streptomycin, or pyrazinamide. These agents will generally be administered according to treatment protocols of the World Health Organization (Geneva, Switzerland) or Center for Disease Control (Atlanta, Ga.). Treatment of Mycobacterium leprae infections may include administration of a composition of the present invention, as well as dapsone, rifampin, clofazimine, or ethionamide according to standard protocols as suggested by the World Health Organization (Geneva, Switzerland) or National Hansen's Disease Center (Carville, La.).

Mycotic infections may also be treated by the methods of the present invention. A wide variety of infections may be treated, such as, e.g., candidiasis (systemic or mucocutaneous), aspergillosis, blastomycosis, chromoblastomycosis, coccidiomycosis, cryptococcosis, histoplasmosis, mucormycosis, paracoccidiodomycosis, pseudallescheriasis, or sporotichosis. Treatment of mycotic infections may be accompanied by administration of anti-fungal agents to the host, such as amphotericin B, flucytosine, ketoconazole, fluconazole, itraconazole, and the like.

Infections by viruses, such as HIV-1, HIV-2, cytomegalovirus, herpesviruses, HTLV-I, HTLV-II, hog cholera virus, distemper virus, feline sarcoma virus, hepatitis viruses, influenza virus, and Dengue virus, may be treated by the methods of the present invention. Adjuvant treatment by anti-viral agents may also be performed. Suitable agents include, e.g., interferon-α, interferon-β, interferon-γ, interferon alfa-2b, cytarabine, acyclovir, idoxuridine, vidarabine, ganciclovir, zidovudine, ribavirin, bromovinyldeoxyuridine, amantidine, foscarnet, dideoxyinosine, dideoxycytidine, azidothymidine, and the like.

Parasitic diseases may be treated by the methods of the present invention. Diseases such as leishmaniasis, pneumocystis infections, giardiasis, trypanosomiasis, malaria, toxoplasmosis, coccidiosis, trichomoniasis, trichinosis, clonorchiasis, echinococcosis, dirofilariasis, and the like may be treated by the present methods. Often, anti-parasitic agents will also be administered to the hosts during treatment.

Vaccination may be augmented by the methods of the present invention. By “augmentation of vaccines”, it is meant that the level and/or duration of complete or partial protection from disease obtained from vaccination is enhanced. Administration of the compositions of the present invention is conjunction with a vaccine may enhance the immune response to the vaccine providing both a higher level of immunity and a prolonged anamnestic response. The peptides may be administered prior to, simultaneously with, or following vaccination. Generally, the peptides will be administered prior to or simultaneously with vaccination.

The methods of the present invention may also be used to treat atopic states. The peptides described above may modulate those components of the immune system responsible for allergic reactions. This may provide an effective treatment for diseases such as acute allergic reactions, chronic urticaria, asthma, and the like.

Hyperimmune states may also be treated by the methods of the present invention. Diseases such as rheumatoid arthritis, systemic lupus erythematosus, Reiter's Syndrome, Psoriasis, ankylosing spondylitis, Sjögren's syndrome, sicca syndrome, mixed connective tissue disorder, multiple sclerosis, diabetes mellitus, and the like may be treated by the methods of the present invention. The immunomodulating properties of the pharmaceutical compositions of the present invention provide a means for re-establishing and maintaining immunological homeostasis. As hyperimmune states cause disease and tissue injury by specific or non-specific immune reactions against tissues, regulation of immune function to a homeopathic state could lessen or prevent autoimmune tissue injury.

The methods of the present invention may provide such a means for immunomodulation. By administering the pharmaceutical compositions of the present invention to a diseased host, the bioactive peptides may re-regulate the immune system by binding to receptors on immunologically-active cells and altering the binding affinity of the receptors to their respective ligands. This re-regulation may restore the normal immunological balance and inhibit autoimmune tissue injury.

The methods of the present invention are also useful for the treatment of graft-versus-host disease. Bone marrow transplant patients may be treated with the compositions of the present invention to lessen the immunoreactivity of the transplanted immunologically-active cells against the host tissue.

Other conditions may also be treated by the methods and compositions of the present invention. For example, dental caries, gingivitis, and periodontitis may be treated. Post-term deliveries may also be accelerated by the compositions and methods of the present invention.

The following examples are offered by way of illustration and not limitation. In the following examples, Glu-Trp is indicated as IM862. IM862 was administered in USP NaCl inhalation solution 0,9% or equivalent at a concentration of 100 μg/ml.

### EXAMPLES

### Example 1

Individuals infected with AIDS are treated with Ile-Trp. This dipeptide is an effective cell mediator, restoring normal immunologic indices, including T-cell functional activity and T4/T8 ratios. Method of Administration: Sterile saline containing the sodium salt of the medication is administered either IM, infralymphatically, or intranasally each day for 5-10 days consecutively every 30 days.

Immunosuppressed individuals who have sustained radiation injuries are treated with Ile-Trp with excellent restoration of immunological indices and models for acquired immune deficiency syndrome. Ile-Trp may thus benefit AIDS infected individuals by reducing the need to use other medications with toxic side effects, and sustain and or support the individuals by reducing the needs to use other medications with toxic side effects, and sustain and or support the individuals immune indices resulting in a reduction of opportunistic infections.

### Example 2

Patients with pyoderma, including furunculitis, cellulitis, and folliculitis, are treated with Ile-Trp with a control group which is not treated with Ile-Trp. Medications are administered either IM or intranasally for 5 consecutive days. Immunological indices are normalized with disappearance of skin manifestations and relapses are prevented after treatment with Ile-Trp. Clinical improvement correlate with immunological indices correction. Administration IM, intranasally, or topically as a sterile saline, solution of medication for a period of 5 to 10 days at a concentration of 1 μg/kg body weight.

### Example 3

A number of patients within the group patients afflicted with furunculitis, pyoderma, cellulitis, and folliculitis are afflicted with acne vulgaris and acne. The immunological indices are corrected and normalized rapidly within the group therapy. The clinical outcome correlates with the correction of immunological indices, and relapses are controlled.

### Example 4

Patients with psoriasis are treated with Ile-Trp and some patients are used as controls. The administration of 100 μg IM or intranasally for a period of 10 days results in the improvement in most of the patients, and total recovery in some of the patients.

### Example 5

Female patients with the various disorders (pelvic inflammatory diseases, cervicitis, vaginitis and various tubo-ovarian and adnexal abscesses) are treated and some patients are used as controls. Ile-Trp is applied IM, intranasally at 100 μg 5 consecutive days or 50 μg intralymphatically for 5 consecutive days in conjunction with conventional therapy. The clinical effect of Ile-Trp expresses the arresting of pain syndrome, the control of body temperature, e.g. reduction of fever, the decrease of duration of conventional treatment. The normalization of immune status correlates with clinical improvements.

### Example 6

Patients treated with Ile-Trp either topically, IM, or intranasally experience marked reduction of recurrence of herpetic lesions, with substantial reduction in the period between outbreaks. Treatment with Ile-Trp in combination with interferon also shows a lessening of lesion outbreaks.

### Example 7

Patients with Herpes Zoster are treated with Ile-Trp in combination with conventional interferon treatment and some control patients with interferon alone. Administration single daily IM or intranasal 100 μg during a period of 10 days results in accelerated regression of foci of herpetic infection. There is noted prevention of relapses, and some healing occurred earlier than control groups. Immunological indices correlates with clinical outcome.

### Example 8

Patients are treated for gingival disease by subcutaneous administration of Ile-Trp in the area of the gingiva. The treatment results in the arresting of gingival disease. Administration of 100 μg IM, subcutaneously, or by electrophoresis (whereby a small voltage charge to the gums results in a rapid transfer of medication through the gum epithelium) results in the arresting of bleeding, more rapid restoration of inflammatory processes, and the decrease of purulent discharge. The treatment results in fewer recurrences and prolongation of normal gums.

### Example 9

The treatment with toothpaste containing Ile-Trp will result in a reduction of dental caries.

### Example 10

Patients with periapical granulomas treated with Ile-Trp are tested. Instillation of 100 μg of Ile-Trp into the foramen at the base of the tooth, or in the composition of the filling paste during 3 days results in the accelerated arrestation of the inflammatory process, reduction in pain, and increases stability of the underlying dental structures as evidenced by x-ray studies.

### Example 11

The use of dental toothpaste containing Ile-Trp will result in the reduction of gingival disease and reduction in dental caries.

### Example 12

The use of Ile-Trp 100 μg IM, intranasally, or intralymphatically controls the advance of lymphangitis.

### Example 13

Patients with acute respiratory disease, including upper airway diseases, such as colds, are treated with Ile-Trp. Administration IM or intranasally 100 μg 3-7 days results in a milder course of the viral infection. Secondary infectious complications are diminished, and the duration of the treatment is also diminished.

### Example 14

Patients are treated with Ile-Trp, administration IM, intranasally, and installation into sinuses with 1 μg/kg dose during a period of 3-10 days results in normalization of nasal breathing, the disappearance of nasal mucous swelling, the arresting of exudates from affect sinuses, and improved general condition and immune status.

### Example 15

Ile-Trp IM or intranasal accompanying conventional therapy (antibiotics) results in accelerated healing of chronic and acute ear infections.

### Example 16

Patients with various eye problems are treated by conventional methods, with one group receiving Ile-Trp in addition to the conventional treatment. Administration of Ile-Trp intra ocularly at 18 μg for 5 consecutive days, or as installation into conjunctival cavity as drops bid for 5 days results in more rapid arresting of the inflammatory process and the increase in visual acuity, and the decrease of duration of treatment.

### Example 17

Patients treated with Ile-Trp and patients in the, control group, are administered medication IM or intranasally 100 μg 5-10 days resulting in accelerated reduction in symptom complexes including joint pain, muscle aches, fevers, chills, and upper respiratory symptoms.

### Example 18

Ile-Trp administration IM or intranasally results in the improved immune parameters, functional activity of lymphocytes and neutrophile, and reduction of post-operative complications and infections associated with bone-marrow compromise, such as, that caused from transplant or radiation exposure.

### Example 19

Patients afflicted with various allergies as described and patients in a control group are treated with Ile-Trp in dose 1 μg/kg IM or intranasally for 5-7 days results in disappearance of allergic reactions.

### Example 20

Patients exposed to massive hemotransfusions during post-operative period are treated with Ile-Trp. The peptide is administered starting from 4-6 day of post-operative period single daily IM or intranasally in doses 100 μg for 5 days. Treated patients do not show clinical manifestation of alloblood rejection while some of the control patients show hemotranfusional reactions.

### Example 21

Ile-Trp is applied in patients treated with antibiotics for various indications who have unfavorable allergological history. Ile-Trp is administered IM or intranasally single daily at 100 μg for 5-10 days. The use of Ile-Trp prevents the arising of allergic reactions or promotes the less severe course in most cases.

### Example 22

Ile-Trp is administered to patients subjected to skin grafting. Ile-Trp is administered IM or intranasally single daily at 50-100 μg for 5 days. In the tested patients the use of Ile-Trp prevents the arising of infections complications and graft rejection.

### Example 23

Ile-Trp is administered to patients suffering from chronic skin diseases caused by antibiotic-resistant staphylococci. Ile-Trp is administered IM in single daily doses of 100 μg for 5 days and intranasally to a different group in the same daily and total dose. In the patients with signs of secondary T-immunodeficiency the staphylococci antibiotic sensitivity to one, few or all antibiotics is increased which then permits one to choose for each patient an effective antibiotic with exclusively high activity against a given pathogen.

### Example 24

Ile-Trp is used in patients with wounds of various origin, type and localization. Ile-Trp is administered IM or topically single daily at 100 μg for 10 days. The use of the dipeptide speeds up (when compared to the control group) significantly wound healing, reduces therapy duration and prevents the development of infectious complications.

### Example 25

Administration of Ile-Trp either intranasally or IM accelerates wound healing, resulting in statistically fewer infections and reduced eschar.

### Example 26

Ile-Trp is applied to patients with bone fractures of various origin, type and localization. Ile-Trp is administered intramuscularly or intranasally single daily at 100 up for 10 days. The use of the dipeptide accelerates essentially (in comparison with the control group) the consolidation of fractures, prevents the development of infectious complications, reduces pain syndrome and treatment duration.

### Example 27

Ile-Trp is prescribed to patients with chronic osteomyelitis of various etiology and localization. Ile-Trp is administered IM or intranasally single daily at 100 μg for 10 days. The use of the peptide renders a pronounced positive influence on clinical course, expressed by a significant decrease of intoxication syndrome and pain syndrome, disappearance of purulent inflammatory manifestations, speeding up of wound healing, reduction of destruction areas, prevention of relapses.

### Example 28

Patients with cutaneous burns are treated with Ile-Trp either IM or intranasally. Accelerated wound healing, diminished frequency of infections, and less eschar are noted in those individuals treated with the peptide.

### Example 29

Patients with frostbite to the extremities are treated with Ile-Trp either IM or intranasally. Rapid healing and restoration of tissue integrity is observed.

### Example 30

Ile-Trp administration either IM or intranasally results in less deformity and scarring evidenced by experience in healing fractures, burns, military accidents, and other injuries to the extremities.

### Example 31

Patients treated with Ile-Trp simultaneously during the administration of chemotherapy experience fewer complications and side effects related to chemotherapy including diminished frequency and intensity of ulcerative lesions, nausea, and other related problems of chemotherapy administration.

### Example 32

Ile-Trp is applied to persons in combination with the anti-flu vaccination delivered by air pressure. The Ile-Trp dose is 50 μg delivered in a single dose for 3 consecutive days. After Ile-Trp use, a significant decrease of sickness rate for a period of 12 months is observed compared to controls who receive flu-vaccination without the peptide.

### Example 33

Ile-Trp is applied in pregnant women with Toxemia of first and second half of pregnancy. Ile-Trp is administered IM and intranasally at 100 μg daily for 5-10 days. It is observed that the BP normalized, and peripheral edema is reduced with normalization of the blood chemistry profile, and the restoration of initially altered immunologic indices.

### Example 34

Ile-Trp is administered to pregnant women. The route of administration is IM or intranasally 100 μg daily for 5-10 days. Signs of clinical improvement are resolution of weakness, dizziness, and increased appetite, and the normalization of the immunological and hematological indices.

### Example 35

Patients with pyelonephritis are treated with the administration of Ile-Trp in a single daily dose of 100 μg for 5-10 consecutive days in combination with conventional therapy which results in reduction of fever, the normalization of urine analysis, and the improvement and resolution of the infection.

### Example 36

Patients with leprosy (Hansen's disease) are treated with Ile-Trp IM or intranasally in single daily doses of 100 μg for 5 days consecutively in additional to conventional therapy. Administration results in resolution of the lesions and prevented relapses, and promotes more rapid healing of specific ulcers.

### Example 37

Patients are studied who have relapsing forms of tropical malaria, moderate to severe, and severe cases with 21 patients in the control group. Ile-Trp is administered at 100 μg single daily doses Im or intranasally for 5-10 days. The results of such treatment are reduction of hepatolineal syndrome, the normalization of hematological and immunological indices, reduction of fever, and prevention of relapses.

### Example 38

Ile-Trp is applied in patients with hemorrhagic Dengue Fever. Ile-Trp is administered IM single daily doses of 100 μg for 5 consecutive days in conjunction with conventional therapy. The results of treatment are reduction in fever, reduction of toxic symptoms, significant decrease in hepato-lineal syndrome.

### Example 39

Patients infected with pulmonary TB are studied and treated. Ile-Trp is administered at 50 to 100 μg every other day during 5 doses total in combination with convention therapy. The results of the treatment several months after treatment reveal the disappearance of toxic symptoms, the reabsorption of infiltrates, and resolution of pulmonary cavities.

### Example 40

Patients, children and adults, with bronchial asthma are studied. Ile-Trp is administered IM single daily doses 1 μg/kg for 5-10 days resulting in less severe clinical symptoms. A significant reduction in bronchial obstruction and laryngotracheitis is noted. The normalization of fever, and the reduction in duration of treatment is noted.

### Example 41

A total 125 patients infected with Shigella dysentery are examined. Ile-Trp is administered IM single doses of 100 μg for 10 consecutive days with resultant normalization of fever, the reduction of toxemia, and the normalization gastrointestinal disorders and symptoms.

### Example 42

About 262 adult patients were treated over a period of 2 months on a daily bases with intramuscular injections of solutions containing 100 μg of Glu-Trp (IM862). These patients were treated for 2 months immediately succeeding exposure to radiation caused from the Chernobyl nuclear accident. As a control, about 19 people exposed to radiation were tested for various blood parameters to establish a baseline.

The results are shown in Table 1 below.

TABLE 1
THE EFFICIENCY OF RADIATION IMMUNODEFICIENCY
CORRECTION TWO MONTHS AFTER IRRADIATION
(X ± m)

Examined groups

Irradiated

| | Healthy | Prior to | After IM862 |
|---|---|---|---|
| Indices | (control) | therapy | therapy |
| Leukocytes, | 5.6 ± 0.8 | 3.5 ± 0.4* | 5.0 ± 1.2** |
| abs | | | |
| Lymphocytes, | 1.98 ± 0.16 | 0.80 ± 0.24* | 1.9 ± 0.4* |
| 2abs | | | |
| CD2-DR+, % | 35.8 ± 0.9 | 21 ± 4* | 30.0 ± 1.2** |
| CD2-DR+, abs | 0.59 ± 0.04 | 0.16 ± 0.04* | 0.55 ± 0.06* |
| CD2, % | 49.3 ± 1.5 | 32 ± 7 | 48.7 ± 1.8** |
| CD2, abs | 0.98 ± 0.09 | 0.55 ± 0.08* | 1.13 ± 0.08** |
| E-RFC, % | 30.2 ± 1.6 | 22.9 ± 1.9* | 27.4 ± 2.4** |
| LMI with | 65.0 ± 2.1 | 120 ± 17* | 90 ± 10** |
| ConA, % | | | |
| CD19, % | 22.0 ± 1.7 | 32 ± 3* | 27 ± 4 |
| CD19, abs | 0.46 ± 0.02 | 0.26 ± 0.06* | 0.51 ± 0.10** |
| IgM, g/l | 1.1 ± 0.4 | 0.87 ± 0.07 | 1.00 ± 0.10 |
| IgG, g/l | 11.1 ± 0.9 | 10.2 ± 2.0 | 10.0 ± 1.0 |
| IgA, g/l | 1.70 ± 0.10 | 1.5 ± 0.4 | 1.49 ± 0.19 |

*statistically significant (P < 0.05) vs. the indices in healthy people;
**statistically significant (P < 0.05) vs. the data obtained prior to immunocorrection with IM862;
abs—cell concentration presented as 10⁹/l;
LMI—leucocyte migration inhibition;
RFC—rosette-forming cells.

### Example 43

The group of patients described in Example 42 were further treated for a period of 36 months and tested again subsequent to the first stage of therapy (after 4 months) and after the second stage of therapy (6 months). The blood parameters are shown in Table 2 below. As can be seen most of the blood parameters were elevated after both the first and second stages of therapy.

TABLE 2
THE PROLONGED IM862 THERAPY TRIALS RESULTS
IN IRRADIATED PATIENTS (X ± m)

| | Interims of examination | | |
|---|---|---|---|
| | | after the 1st | after the 2nd |
| | prior to | stage of | state of |
| Indices | therapy | IM862 use | IM862 use |
| Leukocytes, | 3.5 ± 0.5 | 4.7 ± 0.2* | 5.5 ± 0.3* |
| abs | | | |
| Lymphocytes, | 1.0 ± 0.5 | 1.5 ± 0.4 | 1.9 ± 0.5* |
| abs | | | |
| CD2-DR+, % | 12.8 ± 2.6 | 22.3 ± 0.5 | 29 ± 3* |
| CD2-DR+, abs | 0.13 ± 0.04 | 0.34 ± 0.05* | 0.56 ± 0.08* |
| CD3, % | 24 ± 3 | 35 ± 4* | 46 ± 3* |
| CD3, abs | 0.26 ± 0.05 | 0.49 ± 0.06* | 0.89 ± 0.11* |
| CD4, % | 7.1 ± 1.1 | 19.5 ± 1.7* | 24.1 ± 1.5* |
| CD4, abs | 0.07 ± 0.01 | 0.28 ± 0.03* | 0.45 ± 0.04* |
| CD8, % | 17 ± 3 | 15.4 ± 2.3 | 22.3 ± 2.2* |
| CD8, abs | 0.16 ± 0.04 | 0.23 ± 0.03 | 0.40 ± 0.05* |
| CD19, % | 12.2 ± 1.9 | 15.0 ± 2.8 | 21.1 ± 2.1* |
| CD19, abs | 0.14 ± 0.04 | 0.21 ± 0.06 | 0.39 ± 0.06* |

*statistically significant (P < 0.05) in comparison with the indices prior to therapy;
abs—cell concentration presented as 10⁹/l.

### Example 44

The patients described in Example 42 were tested for blood parameters the first few days after exposure to the radiation of the Chernobyl accident. It could be seen from Table 3 below that response to the treatment was observed even after a few weeks of treatment.

TABLE 3
IM862 INFLUENCE ON IMMUNE STATUS IN EARLY TERMS
AFTER IRRADIATION AFFECTION (X ± m)

| | Examined groups | | |
|---|---|---|---|
| | | Irradiated | |
| | | | After |
| | | Prior to | IM862 |
| Indices | Healthy | therapy | therapy |
| Leukocytes, | 5.7 ± 0.3 | 3.8 ± 0.3* | 6.4 ± 0.8** |
| abs | | | |
| Lymphocytes, | 1.91 ± 0.12 | 1.15 ± 0.0.14* | 2.27 ± 0.16** |
| abs | | | |
| CD2-DR+, % | 30.8 ± 1.1 | 17.6 ± 2.0* | 31 ± 3** |
| CD2-DR+, abs | 0.59 ± 0.04 | 0.20 ± 0.03* | 0.69 ± 0.08** |
| CD2, %, | 50.6 ± 1.6 | 47 ± 4 | 50.9 ± 2.4 |
| CD2, abs | 0.98 ± 0.09 | 0.55 ± 0.08* | 1.13 ± 0.07** |
| E-RFC, % | 29.7 ± 2.5 | 29.8 ± 2.6 | 23.4 ± 2.6 |
| LMI with | 66 ± 4 | 98 ± 9* | 60 ± 7** |
| ConA, % | | | |
| CD19, % | 22.8 ± 2.2 | 27.0 ± 2.8 | 30.5 ± 1.9* |
| CD19, abs | 0.47 ± 0.03 | 0.30 ± 0.05* | 0.68 ± 0.04** |
| IgM, g/l | 1.1 ± 0.4 | 0.51 ± 0.08* | 0.58 ± 0.10* |
| IgG, g/l | 10.1 ± 0.9 | 8.6 ± 1.3 | 9.2 ± 0.7 |
| IgA, g/l | 1.71 ± 0.16 | 2.07 ± 0.20 | 1.11 ± 0.09*,** |
| C3, g/l | 0.57 ± 0.03 | 0.74 ± 0.07 | 0.68 ± 0.04 |

*statistically significant (P < 0.05) in comparison with the indices in healthy people;
**statistically significant (P < 0.05) in comparison with the data obtained prior to IM862 use;
LMI—leukocyte migration inhibition;
abs—cells concentration presented as 10⁹/l.

### Example 45

A number of (36) breast cancer patients were treated with the IM862 by injection of daily dosages of 100 μg (a.i.) The patients had been previously treated with radiation therapy (single doses 2 grad; total dose 45-50 grad). It can be seen from Table 4 below the treatments restore their blood parameter levels.

TABLE 4
IMMUNITY AND NON-SPECIFIC RESISTANCE INDICES
IN BREAST CANCER PATIENTS TREATED WITH IMS62
AFTER RADIOTHERAPY (X ± m)

| | Prior to | After | After IM862 |
|---|---|---|---|
| Indices | radiotherapy | radiotherapy | use |
| Lymphocytes | 1.61 ± 0.18 | 0.79 ± 0.09* | 1.72 ± 0.21** |
| (×10⁹/l) | | | |
| T-lymphocytes | 0.83 ± 0.07+ | 0.32 ± 0.03* | 0.92 ± 0.12** |
| (×10⁹/l) | | | |
| “Active” T- | 0.49 ± 0.06 | 0.19 ± 0.03* | 0.52 ± 0.07** |
| lymphocytes | | | |
| (×10⁹/l) | | | |
| T-helpers | 0.30 ± 0.03 | 0.12 ± 0.01* | 0.39 ± 0.04** |
| (OKT4⁺) | | | |
| T-suppressors | 0.28 ± 0.04 | 0.16 ± 0.02* | 0.21 ± 0.03 |
| (OKT8⁺) | | | |
| (×10⁹/l) | | | |
| OKT4⁺/OKT8⁺ | 1.07 ± 0.09 | 0.75 ± 0.06* | 1.86 ± 0.17** |
| DSH* to | 7.3 ± 0.4 | 2.6 ± 0.2* | 8.7 ± 0.6** |
| tuberculin | | | |
| (mm) | | | |
| LMI^{b} with | 68 ± 4 | 96 ± 7* | 71 ± 5** |
| ConA (%) | | | |
| SI^{c} to IM862 | 1.23 ± 0.15 | 1.19 ± 0.13 | 1.27 ± 0.14** |
| B-lymphocyte | 0.15 ± 0.02 | 0.11 ± 0.01 | 0.17 ± 0.02 |
| (Ig⁺) | | | |
| (×10⁹/l) | | | |
| Phagocytic | 4.3 ± 0.3 | 2.06 ± 0.18* | 3.7 ± 0.2** |
| index | | | |
| Cation | 1.58 ± 0.09 | 1.36 ± 0.08* | 1.49 ± 0.12 |
| C₃-complement | 0.75 ± 0.05 | 0.66 ± 0.04 | 0.68 ± 0.04 |
| (g/l) | | | |

*statistically significant (P < 0.05) vs. the analogous index before radiotherapy;
**statistically significant (P < 0.05) vs. the analogous index after radiotherapy;
^{a}Delayed-Skin Hypersensitivity;
^{b}Leukocyte Migration Inhibition;
^{c}Sensitivity Index.

### Example 46

On peripheral blood of human volunteers in cell cultures were incubated and treated. As can be seen from Table 5 below, after 24 hrs. incubation at concentrations of 1 μg/ml and 100 μg/ml, there was statistically no mutagenic effect in these cultures.

TABLE 5
THE CALCULATION OF CHROMOSOME
STRUCTURAL DAMAGES
IN HUMAN PERIPHERAL BLOOD LYMPHOCYTES

| | | Metaphases | | | |
|---|---|---|---|---|---|
| | | with chromosome | | | |
| Dose of | Number of | structural | | Index of | Level of |
| the | analyzed | aberrations | | reliability | mutagenic |
| medicine | metaphases | # | # | (P) | effect |
| Control | 1000 | 15 | 1.5 | — | — |
| IM862 - 1 μg/ | 1000 | 15 | 1.5 | >0.05 | 0 |
| ml | | | | | |
| IM862 - 1 μg/ | 1000 | 16 | 1.6 | >0.05 | 0 |
| ml | | | | | |

### Example 47

About 263 patients were treated with doses of 100 μg of IM862 introduced intramuscularly on a daily basis over a period of 3 years after exposure to the radiation at the Chernobyl accident. Blood parameters after 3 years of such treatment were restored to the statistical norm prior to their exposure to the radiation. The results are Table 6 below.

TABLE 6

| | | Results of victims examination | | |
|---|---|---|---|---|
| | Statistical | Prior to | After therapy | |
| Indices | norms | therapy | IM862 | Conventional |
| Leukocytes, | 5.2 ± 0.2 | 5.8 ± 0.3 | 5.6 ± 0.4 | 5.5 ± 1.0 |
| abs | | | | |
| Lymphocytes, | 1.96 ± 0.06 | 2.0 ± 0.3 | 2.1 ± 0.3 | 1.8 ± 0.23 |
| abs | | | | |
| CD2-DR+, & | 30.8 ± 1.1 | 15 ± 3* | 32 ± 3** | 18.4 ± 2.5 |
| CD2-DR+, abs | 0.59 ± 0.04 | 0.30 ± 0.06* | 0.66 ± 0.10** | 0.34 ± 0.11 |
| CD3, % | 55.6 ± 1.9 | 67.7 ± 2.7* | 59.2 ± 2.1** | 61 ± 3* |
| CD3, abs | 1.09 ± 0.08 | 1.33 ± 0.05 | 1.21 ± 0.15 | 1.12 ± 0.18 |
| CD4, % | 35.3 ± 2.7 | 36.7 ± 2.6 | 36.2 ± 1.7 | 38 ± 3 |
| CD4, abs | 0.69 ± 0.05 | 0.72 ± 0.05 | 0.74 ± 0.08 | 0.70 ± 0.05 |
| CD8, % | 21.3 ± 0.9 | 29.7 ± 0.9* | 23.2 ± 2.1** | 25.0 ± 2.7** |
| CD8, abs | 0.41 ± 0.03 | 0.56 ± 0.02* | 0.48 ± 0.07** | 0.46 ± 0.06** |
| T4/T8 | 1.64 ± 0.12 | 1.24 ± 0.10* | 1.58 ± 0.04** | 1.52 ± 0.13 |
| IMI, % | 59.7 ± 1.7 | 140 ± 30* | 75 ± 6** | 107 ± 10** |
| B-Ig+, % | 13.8 ± 1.2 | 10.7 ± 0.3 | 11.0 ± 0.3 | 11.2 ± 0.7 |
| B-Ig+, abs | 0.29 ± 0.02 | 0.21 ± 0.01 | 0.23 ± 0.04 | 0.20 ± 0.05 |
| B-IgM+, % | 6.4 ± 0.7 | 3.0 ± 0.3* | 4.1 ± 0.6* | 4.4 ± 0.3 |
| B-IgM+, abs | 0.12 ± 0.01 | 0.062 ± 0.002 | 0.12 ± 0.003 | 0.08 ± 0.004 |
| B-IgG+, % | 4.1 ± 0.5 | 4.7 ± 0.9 | 4.8 ± 0.5 | 4.6 ± 0.5 |
| B-IgG+, abs | 0.078 ± 0.008 | 0.59 ± 0.003 | 0.09 ± 0.007 | 0.08 ± 0.006 |
| B-IgA+, % | 2.2 ± 0.2 | 2.3 ± 0.3 | 1.9 ± 0.3 | 1.98 ± 0.09 |
| B-IgA+, abs | 0.041 ± 0.004 | 0.048 ± 0.006 | 0.04 ± 0.002 | 0.04 ± 0.002 |
| IgM, g/l | 1.15 ± 0.06 | 0.53 ± 0.09 | 1.06 ± 0.06 | 1.03 ± 0.13** |
| IgG, g/l | 11.5 ± 0.5 | 13.2 ± 1.1 | 10.9 ± 1.3 | 11.3 ± 1.2 |
| IgA, g/l | 1.90 ± 0.08 | 0.82 ± 0.25 | 1.2 ± 0.4* | 1.1 ± 0.3 |

*statistically significant (P < 0.05) vs. the indices in healthy persons;
**statistically significant (P < 0.05) vs. the data obtained prior to therapy;
abs—cell concentration presented as 10⁹/l;
LMI—leucocyte migration inhibition.

### Example 48

The patients described above in Example 47 were tested for blood parameters after 6 months of treatment immediately following exposure to the radiation caused by the Chernobyl accident. The results in Table 7 below show that after 6 months those treated with IM862 showed improvement over those patients who were not treated.

TABLE 7
IM862 USE EFFICIENCY IN ACUTE RADIATION
SICKNESS (6 MONTHS AFTER ACCIDENT)

| | Healthy | Patients (suffered in accident) | | |
|---|---|---|---|---|
| | people | | After therapy | |
| | (statistic | Prior to | Without | |
| Indices | normal) | therapy | IM862 | With IM862 |
| Lymphocytes, % | 33.9 ± 1.2 | 32.9 ± 2.4 | 29.2 ± 2.0 | 30.0 ± 1.8 |
| Lymphocytes, | 1.96 ± 0.06 | 1.49 ± 0.14* | 1.39 ± 0.13 | 1.52 ± 0.12* |
| abs | | | | |
| CD2, % | 53.6 ± 1.9 | 38.7 ± 2.7* | 32 ± 3* | 49 ± 3** |
| CD2, abs | 1.05 ± 0.05 | 0.56 ± 0.04* | 0.44 ± 0.04 | 0.75 ± 0 ± 0.055*,** |
| CD2-DR+, % | 30.8 ± 1.1 | 18.9 ± 1.6* | 19.7 ± 1.2* | 20.8 ± 1 ± 0.66*,** |
| CD2-DR+, abs | 0.59 ± 0.04 | 0.30 ± 0.25* | 0.28 ± 0.02* | 0.31 ± 0 ± 0.022*,** |
| CD3, % | 55.6 ± 1.9 | 39.0 ± 2.4* | 37 ± 5* | 53.4 ± 1.8** |
| CD3, abs | 1.09 ± 0.08 | 0.58 ± 0.04* | 0.51 ± 0.03 | 0.82 ± 0.04** |
| CD4, % | 35.3 ± 2.7 | 20.3 ± 1.3* | 18.9 ± 1.3* | 32.6 ± 1.4** |
| CD4, abs | 0.69 ± 0.05 | 0.30 ± 0.03* | 0.26 ± 0.03* | 0.50 ± 0.04* |
| CDS, % | 21.2 ± 0.9 | 19.5 ± 1.5 | 17.5 ± 1.6 | 21.2 ± 1.8 |
| CDS, abs | 0.41 ± 0.03 | 0.29 ± 0.03 | 0.24 ± 0.03 | 0.32 ± 0.03 |
| T4/T8 | 1.64 ± 0.12 | 1.04 ± 0.04* | 1.08 ± 0.10* | 1.54 ± 0.11** |
| LMI | 59.7 ± 1.7 | 106 ± 6* | 107 ± 6* | 72.7 ± 4.5*,** |
| CD19, % | 25.00 ± 0.12 | 18.2 ± 2.1* | 23 ± 3 | 26.7 ± 2.1 |
| CD19, abs | 0.49 ± 0.04 | 0.27 ± 0.03* | 0.21 ± 0.05* | 0.041 ± 0.03** |
| B-Ig+, % | 13.8 ± 1.2 | 15.8 ± 1.3 | 16.2 ± 1.7 | 19.0 ± 1.3*,** |
| B-Ig+, abs | 0.29 ± 0.02 | 0.23 ± 0.03 | 0.23 ± 0.04 | 0.29 ± 0.03 |
| B-IgM+, % | 6.4 ± 0.7 | 6.3 ± 0.8 | 5.4 ± 0.5 | 8.8 ± 0.7 |
| B-IgM+, abs | 0.12 ± 0.01 | 0.09 ± 0.01* | 0.08 ± 0.01* | 0.13 ± 0.02 |
| B-IgG+, % | 4.1 ± 0.5 | 7.8 ± 0.9* | 7.1 ± 0.5* | 6.4 ± 0.5* |
| B-IgG+, abs | 0.082 ± 0.008 | 0.098 ± 0.007 | 0.104 ± 0.008 | 0.100 ± 0.007 |
| B-IgA+, % | 2.20 ± 0.20 | 1.80 ± 0.15* | 1.70 ± 0.20* | 1.8 ± 0.3 |
| B-IgA+, abs | 0.038 ± 0.004 | 0.033 ± 0.004 | 0.024 ± 0.003 | 0.030 ± 0.002 |
| IgM, g/l | 1.15 ± 0.06 | 1.14 ± 0.08 | 1.20 ± 0.07 | 1.07 ± 0.09 |
| IgG, g/l | 11.5 ± 0.5 | 11.9 ± 1.0 | 11.7 ± 0.9 | 10.9 ± 1.1 |
| IgA, g/l | 1.9 ± 1.0 | 1.6 ± 0.8 | 1.6 ± 0.8 | 1.8 ± 0.9 |

*statistically significant (P < 0.05) vs. the indices in healthy people;
**statistically significant (P < 0.05) vs. the data obtained before immunocorrection;
abs—cell concentration presented as 10⁹/liter;
LKI—leucocyte migration inhibition.

### Example 49

A group of 452 persons were treated with daily dosages of 100 μg of IM862 administered intramuscularly over a period of 5-10 days and compared with a random group (250 persons) (not similarly treated) as a control. The cases of respiratory diseases and influenza were recorded for both groups. As can be seen from Tables 8 and 9 below, the untreated group had a greater occurrence of the diseases and sicknesses, hospitalization or disablement than the group treated with IM862.

TABLE 8
IM862 CLINICO-EPIDEMIOLOGICAL PREVENTIVE
EFFICIENCY IN ARD AND FLU

| | |
|---|---|
| Group of | |
| observation | Index of |

| Indices | IM862 | Control | efficiency |
|---|---|---|---|
| Sickness rate | 9.8 | 30.4 | 3.1 |
| per 100 | | | |
| persons/month | | | |
| Pneumonia rate/ | 0.20 | 0.50 | 2.5 |
| 100 | | | |
| persons/month | | | |
| Need in | 30.6 | 44.9 | 1.7 |
| hospitalization | | | |
| Average term of | 6.2 | 8.8 | 1.4 |
| hospitalization, | | | |
| days | | | |
| Rate of | 3.9 | 13.8 | 3.5 |
| lingering and | | | |
| complicated | | | |
| cases, % | | | |
| The same index | 9.8 | 26.2 | 2.7 |
| in-patients, % | | | |
| Number of | 4.1 | 7.0 | 1.7 |
| temporary | | | |
| disablement | | | |
| cases/100 | | | |
| persons/month | | | |
| Number of | 26.5 | 57.6 | 2.2 |
| temporary | | | |
| disablement | | | |
| days/100 | | | |
| persons/month | | | |

TABLE 9
THE DYNAMICS OF ARD AND INFLUENZA
RATE IN GROUPS OF OBSERVATION
BY MONTHS FROM THE BEGINNING
OF INVESTIGATION

| Indices | Groups | 1st month | 2nd month | 3rd month | 4th month |
|---|---|---|---|---|---|
| Sickness | IM862 | 9.6 | 11.3 | 9.4 | 11.0 |
| rate/100 | Control | 28.6 | 33.4 | 28.7 | 30.6 |
| persons/month | | | | | |
| Need in | IM862 | 27.0 | 27.1 | 28.3 | 28.3 |
| hospitalization, | Control | 41.2 | 50.8 | 48.8 | 39.0 |
| % | | | | | |
| Average term | IM862 | 6.2 | 6.2 | 6.2 | 7.0 |
| of | Control | 9.3 | 8.4 | 10.2 | 11.0 |
| hospitalization, | | | | | |
| days | | | | | |
| Number of | IM862 | 3.8 | 7.3 | 3.4 | 3.6 |
| temporary | control | 6.9 | 10.2 | 7.5 | 5.6 |
| disablement | | | | | |
| cases/100 | | | | | |
| persons/month | | | | | |
| Number of | IM862 | 24.0 | 48.6 | 14.3 | 26.3 |
| temporary | control | 47.9 | 82.0 | 51.4 | 42.8 |
| disablement | | | | | |
| days/100 | | | | | |
| persons/month | | | | | |

### Example 50

In separate studies, a total of 21 AIDS infected individuals have been studied, including full-blown syndrome, prodromal, and pre-AIDS afflicted individuals who were treated with Thympentin. Thympentin and TPI are thymic gland peptide extracts previously well characterized. Comparative studies between TPI and IM862 reveal that IM862 is a far more effective cell mediator, restoring normal immunologic indices, including T-cell functional activity and T4/T8 ratios. Method of Administration: Sterile saline containing the sodium salt of the medication is administered either IM, infralymphatically, or intranasally each day for 5-10 days consecutively every 30 days.

Immunosuppressed individuals who have sustained radiation injuries were treated with IM862 with excellent restoration of immunological indices and models for acquired immune deficiency syndrome. IM862 may thus benefit AIDS infected individuals by reducing the need to use other medications with toxic side effects, and sustain and or support the individuals by reducing the needs to use other medications with toxic side effects, and sustain and or support the individuals immune indices resulting in a reduction of opportunistic infections.

### Example 51

A total of 159 patients having pyoderma were treated with IM862, including furunculitis, cellulitis, and folliculitis, with an additional control group consisting of 25 patients who were not treated with IM862. Medications were administered either IM or intranasally for 5 consecutive days. Immunological indices were normalized with disappearance of skin manifestations and relapses were prevented after treatment with IM862. Clinical improvement correlated with immunological indices correction. Administration IM, intranasally, or topically as a sterile saline solution of medication for a period of 5 to 10 days at a concentration of 1 μg/kg body weight.

### Example 52

A number of patients within the group of 159 patients afflicted with furunculitis, pyoderma, cellulitis, and folliculitis were afflicted with acne vulgaris and acne. The immunological indices corrected and normalized rapidly within the group therapy. The clinical outcome correlated with the correction of immunological indices, and relapses were controlled.

### Example 53

A total or 30 patients were treated with psoriasis and 30 patients were used as controls and were untreated with IM862. All patients had at a least 5 year history of no unsuccessful treatment. The administration of 100 μg IM or intranasally for a period of 10 days resulted in the improvement in 7% of the patients, significant improvement in 60% of patients, and total recovery in 33% of the patients.

### Example 54

A total of 46 female patients with the various disorders (pelvic inflammatory diseases, cervicitis, vaginitis and various tubo-ovarian and adnexal abscesses) were treated and 50 patients were used as controls IM862 was applied IM, intranasally at 100 μg 5 consecutive days or 50 μg intralymphatically for 5 consecutive days in conjunction with conventional therapy. The clinical effect of IM862 expressed the arresting of pain syndrome, the control of body temperature, e.g. reduction of fever, the decrease of duration of conventional treatment. The normalization of immune status correlated with clinical improvements.

### Example 55

Patients treated with IM862 either topically, IM, or intranasally experienced marked reduction of recurrence of herpetic lesions, with substantial reduction in the period between outbreaks. In one study, individuals who experienced 7-10 outbreaks per year experienced less than one outbreak per year after treatment with IM862 in combination with interferon.

### Example 56

A total of 37 patients with Herpes Zoster were treated with IM862 in combination with conventional interferon treatment and 25 control patients with interferon alone. Administration single daily TM or intranasal 100 μg during a period of 10 days resulted in accelerated regression of foci of herpetic infection. There was noted prevention of relapses, and healing occurred on the average 40% earlier than control groups. Immunological indices correlated with clinical outcome.

### Example 57

Patients were treated for gingival disease by subcutaneous administration of IM862 in the area of the gingiva. The treatment resulted in the arresting of gingival disease. Approximately 80 patients were studied with disease and treated and an equal number were treated conventionally without IM862 for control purposes. Administration of 100 μg IM, subcutaneously, or by electrophoresis (whereby a small voltage charge to tho gums results in a rapid transfer of medication through the gum epithelium) resulted in the arresting of bleeding, more rapid restoration of inflammatory processes, and the decrease of purulent discharge. The treatment resulted in fewer recurrences and prolongation of normal gums. It was also noted that normalization of immunologic indices was achieved with normal coagulation.

### Example 58

The treatment with toothpaste containing IM862 will result in a reduction of dental caries.

### Example 59

A total of 46 patients with periapical granulomas and 28 patients with the same disease not treated with IM862 were used for controls. Instillation of 100 μg of IM862 into the foramen at the base of the tooth, or in the composition of the filling paste during 3 days resulted in the accelerated arrestation of the inflammatory process, reduction in pain, and increased stability of the underlying dental structures as evidenced by x-ray studies.

### Example 60

The use of dental toothpaste containing IM862 will result in the reduction of gingival disease and reduction in dental caries.

### Example 61

The use of IM862 100 μg IM, intranasally, or intralymphatically controls the advance of lymphangitis.

### Example 62

A total of 186 patients with acute respiratory disease, including upper airway diseases, such an colds, were treated with IM862 and 87 patients who were not treated with IM862 were used as controls. Administration IM or intranasally 100 μg 3-7 days resulted in a milder course of the viral infection. There was noted a decrease in the specific signs of upper respiratory infections such rhinorrhea, sore throat, fever, muscle aches, headaches, and ear pain. Secondary infectious complications were diminished, and the duration of the treatment was also diminished.

### Example 63

A total of 51 patients were treated with IM862 with 24 patient controls, administration IM, intranasally, and installation into sinuses with 1 μg/kg dose during a period of 3-10 days resulted in normalization of nasal breathing, the disappearance of nasal mucous swelling, the arresting of exudates from affect sinuses, and improved general condition and immune status. The decrease of treatment duration up to 1.7 times compared to controls.

### Example 64

IM862 IM or intranasal accompanying conventional therapy (antibiotics) results in accelerated healing of chronic and acute ear infections.

### Example 65

A total of 41 patients with various eye problems as described and 36 patients in control studies were treated by conventional methods with the first group receiving IM862 in addition to the conventional treatment. Administration of IM862 intra ocularly at 18 μg for 5 consecutive days, or as installation into conjunctival cavity as drops bid for 5 days resulted in more rapid arresting of the inflammatory process and the increase in visual acuity, and the decrease of duration-of treatment.

### Example 66

A total of 156 patients treated with IM862 and 82 patients in the control study, were administered, medication IM or intranasally 100 μg 5-10 days resulting in accelerated reduction in symptom complexes including joint pain, muscle aches, fevers, chills, and upper respiratory symptoms.

### Example 67

A total of 263 patients and 18 control patients sustained exposure to radiation injury. IM862 was administered IM and/or intranasally 100 μg for 10 days. A repeated course may be proscribed on the basis of immunological indices, and averages every 4 to 6 months. The results of the treatment are restoration of normal or near normal immune indices with functional activity in the majority of all cases studied. There was an arresting of asthenic syndrome, and an arresting of the somatic pathological exacerbations and reduction of opportunistic infections.

### Example 68

IM862 administration IM or intranasally results in the improved immune parameters, functional activity of lymphocytes and neutrophils, and reduction of post-operative complications and infections associated with bone-marrow compromise, such as, that caused from transplant or radiation exposure.

### Example 69

A total of 29 patients afflicted with various allergies as described and 17 patients in the control group were treated with IM862 in dose 1 μg/kg IM or intranasally for 5-7 days resulted in disappearance of allergic reactions.

### Example 70

A total of 76 patients with 72 patients in control exposed to massive hemotransfusions during post-operative period were treated with IM862. IM862 was administered starting from 4-6 day of post-operative period single daily IM or intranasally in dose 100 μg for 5 days. None of studies patients showed clinical manifestation of alloblood rejection while in 17% of control patients the adverse hemotranfusional reactions were observed.

### Example 71

IM862 was applied in 76 patients treated with antibiotics for various indications who had unfavorable allergological history. Control group comprised 43 patients. IM862 was administered IM or intranasally single daily at 100 μg for 5-10 days. In the majority of case the use of IM862 prevented the arising of allergic reactions or promoted the less severe course of them while in the control group in 70% of patients the pronounced signs of drug intolerance was marked.

### Example 72

IM862 was administered to 17 patients subjected to skin grafting. The control group comprised 27 patients. IM862 was administered IM or intranasally single daily at 50-100 μg for 5 days. In all the patients the use of IM862 prevented the arising of infections complications and graft rejection. In control group the manifestations of rejection were determined in 8 patients.

### Example 73

IM862 was administered to 52 patients suffered from chronic skin diseases caused by antibiotic-resistant staphylococci. 47 patients with the same pathology but not treated with the immunomodulator were the control group. IM862 was administered IM to 27 patients single daily at 100 μg for 5 days and intranasally to 25 patients in the same daily and total dose. The differences between these methods of application were not noticed. In all the patients with signs of secondary T-immunodeficiency the staphylococci antibiotic-sensitivity to one, few or all antibiotics has been increased sharply (more than 100-fold) what permitted further to choose for each patient the antibiotic with exclusively high activity against pathogen. As a whole, within the examined group of patients the reliable decease of MIC of all studied antibiotics has been marked. The proposed treatment regiment permitted to obtain the complete recovery in 27 patients, significant improvement—in 8 patients and moderate improvement—in 1 patient.

### Example 74

IM862 was used in 37 patients with wounds of various origin, type and localization. The control group comprised 24 patients. IM862 was administered IM or topically single daily at 100 μg for 10 days. The use of IM862 speeded up (when compared to the control group) significantly wound healing, reduced therapy duration and prevented the development of infectious complications.

### Example 75

Administration of IM862 either intranasally or IM accelerates wound healing, resulting in statistically fewer infections and reduced eschar.

### Example 76

IM862 was applied to 44 patients with bone fractures various origin, type and localization. The control group comprised 28 patients. IM862 was administered intramuscularly or intranasally single daily at 100 up for 10 days. The use of IM862 accelerated essentially (in comparison with the control group) the consolidation of fractures, prevented the development of infectious complications, reduced pain syndrome and treatment duration.

### Example 77

IM862 was prescribed to 176 patients with chronic osteomyelitis of various etiology and localization. The control group comprised 88 patients. IM862 was administered IM or intranasally single daily at 100 μg for 10 days. The use of IM862 rendered the pronounced positive influence on clinical course what expressed in significant decrease of intoxication syndrome and pain syndrome, disappearance of purulent inflammatory manifestations, speeding up of wound healing, reduction of destruction areas, prevention of relapses.

### Example 78

A total of 23 patients with cutaneous burns were treated with IM862 either IM or intranasally with 14 patients for control treated conventionally. Accelerated wound healing, diminished frequency of infections, and less eschar was noted in those individuals created with IM862.

### Example 79

A total of 17 patients with frostbite to tho extremities where treated with IM862 either IM or intranasally with 11 patient controls. The rapid healing and restoration of tissue integrity was observed.

### Example 80

IM862 administration either IM or intranasally results in less deformity and scarring evidenced by experience in healing fractures, burns, military accidents, and other injuries to the extremities.

### Example 81

Experimental data supports the finding that IM862 administered IM, intranasally, or ocular installation results in restoration and regeneration of corneal epithelium with fewer infections and complications related to eschar.

### Example 82

A total of 246 patients with various forms of cancer, and 158 controls after radiation and chemo-therapy, where IM862 was administered in single 100 μg daily dose for 10 days experienced normalization of immunological indices, the prevention of postoperative infections, the prevention of upper respiratory infections, and prevention of exacerbations of various secondary complications such as gastritis, cholecystitis, etc. If it vas determined necessary based on immunological indices, the treatment regimen was repeated in 4-6 months.

### Example 83

Patients treated with IM862 simultaneously during the administration of chemotherapy experienced fewer complications and side effects related to chemotherapy including diminished frequency and intensity of ulcerative lesions, nausea, and other related problems of chemotherapy administration.

### Example 84

Experimental models support the fact that administration of IM862 prophylactically results in diminished frequency of spontaneous tumorigenesis.

### Example 85

IM862 was applied to 268 persons in combination with the anti-flu vaccination. The control group comprised 197 persons. The vaccination was delivered by air pressure. The IM862 dose was 50 μg delivered in a single dose for 3 consecutive days. After IM862 use, it was observed the significant decrease of sickness rate for a period of 12 months compared to controls who received flu-vaccination without IM862. In the event of flu, the course of the infection was noted to be less severe and the recovery more rapid when compared to controls.

### Example 86

IM862 was applied in 97 pregnant women with Toxemia of first and second half of pregnancy. The control group comprised 54 patients. IM862 was administered IM and intranasally at 100 μg daily for 5-10 days. Under the influence of IM862, it was observed that the BP normalized, and peripheral edema was reduced with normalization of the blood chemistry profile, and the restoration of initially altered immunologic indices.

### Example 87

IM862 was administered to 34 pregnant women with 27 pregnant women for control. The route of administration is IM or intranasally 100 μg daily for 5-10 days. Signs of clinical improvement were resolution of weakness, dizziness, and increased appetite, and the normalization of the immunological and hematological indices. It was also noted that there was a decrease in fetal hypoxia.

### Example 88

A total of 19 post-term women and 48 women post-term in the control study were treated. The administration of 100 pg of IM862 IM or intranasally over 3-5 days resulted in the effacement of the cervix with thinning at the cervix and the decent of the fetus, with subsequent spontaneous normal delivery.

### Example 89

A total of 27 patients with pyelonephritis and 19 control patients with pyelonephritis were treated with the administration of IM862 single daily dose of 100 μg for 5-10 consecutive days in combination with conventional therapy resulted in reduction of fever, the normalization of urine analysis, and the improvement and resolution of the infection. The normal course of delivery in those women treated with IM862 was without complications.

### Example 90

A total of 45 patients with leprosy (Hansen's disease) and 27 infected individuals were treated. IM862 was administered im or intranasally in single daily doses of 100 μg for 5 days consecutively in additional to conventional therapy. The patients studied had previous documented resistance to treatment by conventional methods. IM862 administration resulted in resolution of the lesions and prevented relapses, and promoted more rapid healing of specific ulcers. The immunologic indices were normalized.

### Example 91

IM862 was administered to 84 young sportsmen. The control group consisted of 44 persons. The IM862 was administered intranasally single dose 1 μg/kg during 3 days. The use of IM862 resulted in the reduction of upper respiratory infections and rates of illness 4 fold. In the event of infection, it was noted that the infections was less severe without complications, and the clinical improvement was accompanied by the normalization of immunological indices.

### Example 92

A total of 33 patients were studied with patients who had relapsing forms of tropical malaria, moderate to severe, and severe cases with 21 patients in the control group. IM862 was administered at 100 μg mingle daily doses IM or intranasally for 5-10 days. The results of such treatment was reduction of hepatolineal syndrome, the normalization of hematological and immunological indices, reduction of fever, and prevention of relapses.

### Example 93

IM862 was applied in 27 persons with the goal to increase the resistance to excessive solar radiation, in the conditions of hot marine climates. The control group comprised 24 persons. The administration was intranasally 100 μg for 3 days. The use of IM862 prevented the occurrence of upper respiratory infections In the treated group relative to the control group. There was also noted suppression of their immunologic indices.

### Example 94

IM862 was applied in 21 patients with hemorrhagic Dengue Fever, and 28 patients served as controls. IM862 was administered IM single daily doses of 100 μg for 3 consecutive days in conjunction with conventional therapy. The results of treatment were reduction in fever. reduction of toxic symptoms, significant decrease in hepatolineal syndrome. It was also noted that the muscular and bone pain experienced typically was reduced, and the immunological indices were normalized.

### Example 95

A total of 48 patients infected and 34 infected controls were examined and treated with administration of IM862 100 μg IM or intranasally for 5-10 days resulting in normalization of fever, the reduction of toxic symptoms, and the resolution of icterus (jaundice). The hematological and immunological indices were normalized.

### Example 96

A total of 36 patients infected and 24 patients infected were controls. Administration of IM862 In 100 μg IM or intranasally for 5-10 days resulted in the reduction of fever, more rapid reduction of toxic symptoms, and the restoration of immunologic indices.

### Example 97

A total of 37 patients infected with pulmonary TB and 26 patients infected as controls were studied and treated. IM862 was administered at 50 to 100 μg every other day during 5 doses total in combination with convention therapy. The results of the treatment 2 months after the course of IM862 revealed the disappearance of toxic symptoms, the reabsorption of infiltrates, and resolution of pulmonary cavities. The disappearance of TB bacilli as noted in the sputum. The restoration of initially decreased immune indices was also noted.

### Example 98

A total of 37 patients, children and adults, with bronchial asthma and 28 similar patients as controls were studied. IM862 was administered IM single daily doses 1 μg/kg for 5-10 days resulting in less severe clinical symptoms. The significant reduction in bronchial obstruction and laryngotracheitis was noted. The normalization of fever, and the reduction in duration of treatment was noted. In some of the patients it was possible to avoid steroids in the conventional commitment treatment course. In the allowing year observation there was noted a decrease in the incidence of bronchial asthma 4.2 fold. In more than half of the patients the disappearance of drug and food allergy manifestations was noted.

### Example 99

A total 125 patients with 53 patients for control infected with Shigella dysentery were examined. IM862 was administered IM single doses of 100 μg for 10 consecutive days with resultant normalization of fever, the reduction of toxemia, and the normalization gastrointestinal disorders and symptoms. Bacterial shedding in the GI track was observed to cease, and tho immunological indices were normalized.

### Example 100

A total of 12 patients who had been thymectomized were treated with IM862. Prior to therapy these individuals had experienced frequent serious infections including upper respiratory infections. IM862 was administered in a single dose 100 μg daily for 10 days and repeated every 4-6 months. The normalization of immunologic indices was observed, and there was reduction of infectious disorders including cutaneous infections and other chronic exacerbations.

### Example 101

A total of 39 patients were studied with 27 patient controls. IM862 was administered IM or intranasally at 100 up for 5-10 days to the study group or patients with the resulting reduction of fever, decrease in toxic symptoms, the reduction of musculoskeletal pain, and the reduction or disappearance of jaundice. Immunological indices were normalized.

### Example 103

The use of IM862 as an ingredient or applicant with cosmetics provides for a less allergenic cosmetic with fewer allergic reactions.

### Example 104

To prevent and treat bronchopneumonia, hypotrophy or nitrate intoxication in cattle or swine, an intramuscular dose of 1 microgram/kg body weight of the dipeptide is administered: cycle of 4-6 months. To prevent and treat viral diseases, Marek's disease or hypotrophy in poultry, a mist is delivered to the incubator habitat of the poultry in a concentration of dipeptide of about 200/microgram/cu. meter over 1-3 day cycles.

### Example 105

This example demonstrates the use of compositions containing Glu-Trp to prolong tissue graft survival. Grafts in animals that received Glu-Trp had prolonged survival compared to animals not receiving Glu-Trp.

Skin grafts observed for 14 days in host mice allograft recipients. Viability of grafting was observed for 10 mice in each group administering IM862, Thymalin or saline, daily for 5 treatments prior to grafting. The results are shown in Table 10.

TABLE 10

| | | GRAFT | |
|---|---|---|---|
| | | REJECTION | |
| | | TIME | |
| MEDICINE | N | (DAYS) | P |
| IM862 0.01 | 10 | 8.3 ± 0.4 | — |
| mg/kg | | | |
| Thymalin 1.0 | 10 | 5.9 ± 0.4 | >0.05 |
| mg/kg | | | |
| Control (saline) | 10 | 5.6 ± 0.3 | >0.05 |

TABLE 11

| | 20 days after irradiation | | |
|---|---|---|---|
| | | | Irradiated + |
| | | | IM862 |
| | | | 0.01 mg/kg |
| Thymic | | | daily for 5 |
| Indices | Healthy | Irradiated | days |
| Number of animals | 8 | 7 | 7 |
| Weight coefficient | 0.86 ± 0.06 | 0.63 ± 0.6^{x} | 0.81 ± 0.8^{xx} |
| g/kg | | | |
| Lymphocytes | 481.4 ± 21.3 | 254.1 ± 32.7^{x} | 423.3 ± 40.4^{xx} |
| ×10³/mg | | | |
| T-lymphocytes % | 78.6 ± 8.1 | 28.3 ± 2.4^{x} | 81.3 ± 9.3 |
| ×10³/mg | 378.5 ± 26.4 | 71.4 ± 5.6^{x} | 342.8 ± 30.4^{xx} |
| “Active” | 60.3 ± 5.1 | 41.6 ± 5.7^{x} | 57.6 ± 6.2^{xx} |
| T-lymphocytes % | 290.2 ± 27.6 | 105.7 ± 12.6^{x} | 242.6 ± 26.8^{xx} |
| ×10³/mg | | | |
| B-lymphocytes % | 0.9 ± 0.07 | 1.6 ± 0.1^{x} | 0.9 ± 0.08^{xx} |
| ×10³/mg | 4.8 ± 0.31 | 4.1 ± 0.5 | 3.7 ± 0.4 |
| O-lymphocytes % | 20.5 ± 1.1 | 70.4 ± 5.3 | 17.9 ± 0.8 |
| ×10³/mg | 98.6 ± 6.3 | 178.8 ± 19.3^{x} | 75.4 ± 8.6^{xx} |

^{x}statistically significant (p < 0.05) as compared to healthy (normal) animals.
^{xx}statistically significant (p < 0.05) as compared to irradiated animals

TABLE 12

| | 20 days after Irradiation | | |
|---|---|---|---|
| | | | Irradiated + |
| | | | IM862 |
| | | | 0.01 mg/kg |
| Lymph Node | | | daily for 5 |
| Indices | Healthy | Irradiated | days |
| Number of animals | 8 | 7 | 7 |
| Weight coefficient | 0.29 ± 0.02 | 0.31 ± 0.03 | 0.31 ± 0.03 |
| g/kg | | | |
| Lymphocytes ×10³/mg | 251.6 ± 18.4 | 108.5 ± 14.6^{x} | 242.5 ± 31.3^{xx} |
| T-lymphocytes % | 13.4 ± 0.9 | 16.2 ± 0.9 | 12.3 ± 0.9 |
| ×10³/mg | 32.8 ± 3.0 | 17.6 ± 1.5^{x} | 29.9 ± 2.2^{xx} |
| “Active” | 7.2 ± 0.6 | 8.4 ± 0.8 | 10.3 ± 1.4 |
| T-lymphocytes % | 18.1 ± 1.1 | 9.1 ± 0.9^{x} | 24.9 ± 3.7^{xx} |
| ×10³/mg | | | |
| B-lymphocytes % | 22.2 ± 3.4 | 7.9 ± 0.7^{x} | 3.5 ± 0.6^{x} |
| ×10³/mg | 56.1 ± 4.1 | 7.2 ± 0.9^{x} | 9.2 ± 0.9^{x} |
| O-lymphocytes % | 64.4 ± 0.5 | 76.6 ± 10.6 | 84.4 ± 12.3 |
| ×10³/mg | 162.0 ± 12.3 | 83.1 ± 9.6^{x} | 204.6 ± 33.4^{xx} |

^{x}statistically significant (p < 0.05) as compared to healthy (normal) animals.
^{xx}statistically significant (p < 0.05) as compared to irradiated animals.

TABLE 13

| | 20 days after irradiation | | |
|---|---|---|---|
| | | | Irradiated |
| | | | + IM862 |
| | | | 0.01 mg/kg |
| Splenic | | | daily for 5 |
| Indices | Healthy | Irradiated | days |
| Number of animals | 8 | 7 | 7 |
| Weight coefficient | 1.30 ± 0.1 | 1.27 ± 0.1 | 1.26 ± 0.9 |
| g/kg | | | |
| Lymphocytes ×10³/mg | 409.3 ± 25.6 | 272.7 ± 46.7^{x} | 260.4 ± 32.3^{xx} |
| T-lymphocytes % | 9.8 ± 0.8 | 11.5 ± 0.8 | 17.4 ± 1.2 |
| ×10³/mg | 40.4 ± 3.3 | 30.1 ± 2.9^{x} | 45.4 ± 4.1^{xx} |
| “Active” | 6.4 ± 0.5 | 7.3 ± 0.7 | 11.1 ± 2.0 |
| T-lymphocytes % | 26.1 ± 1.9 | 19.9 ± 3.4^{x} | 28.9 ± 3.9^{xx} |
| ×10³/mg | | | |
| B-lymphocytes % | 17.7 ± 1.2 | 24.3 ± 1.8 | 27.1 ± 2.6 |
| ×10³/mg | 72.5 ± 5.4 | 66.5 ± 8.47 | 81.6 ± 9.3 |
| O-lymphocytes % | 72.5 ± 6.3 | 64.7 ± 8.1 | 57.4 ± 6.7 |
| ×10³/mg | 296.7 ± 12.4 | 176.4 ± 19.3^{x} | 149.4 ± 15.9^{x} |

^{x}statistically significant (p < 0.05) as compared to healthy (normal) animals.
^{xx}statistically significant (p < 0.05) as compared to irradiated animals.

### Example 105

This example describes the effect of administration of a composition of the present invention on immune function of animals following radiation exposure. Administration of IM862 enhanced immunological restoration.

The influence of IM862 on the immune system of the body after radiation exposure was assessed in an experiment conducted on 96 male guinea pigs of mass 250-300 g. Seventy-two of the animals were irradiated with total radiation dose was 1 Gr. A day after irradiation one group of animals (24 guinea pigs) began receiving intramuscular administrations of IM862 in a dose of 0.01 mg/kg (in 0.5 ml of saline solution) daily for 5 days. The selected doses were optimal in stimulating an immune response to ram erythrocytes. Control irradiated and non-irradiated animals (24 guinea pigs to each group) were given saline solution per an analogous schedule. At 5, 10, and 20 days after irradiation the animals were destroyed (8 guinea pigs from each group). The thymus, spleen, and lymph nodes of each animal were extracted and their weight coefficients computed. The number of karyocytes, T-, B-, and O-lymphocytes in each organ was determined. Also, the functional activity of blood lymphocytes in the leukocyte migration inhibition reaction to Con A was assessed and the content of cation proteins in the neutrophils was determined. The results of these investigations are provided in the Tables below.

As noted in Tables 11-13 above, the ionizing radiation caused a decline in the quantity of karyocytes, T-, and B-lymphocytes in the lymphoid organs of irradiated animals. Significant alteration of the cellular composition of organs was especially noted in the thymus and lymph nodes. Functional activity of blood lymphocytes and neutrophils of irradiated guinea pigs was suppressed. This was evidenced by an increase of 1.5-2 times in the percent of leukocyte migration and a drop by 20-35% in the quantity of cation proteins in the neutrophils.

Administration of IM862 limited the decline of lymphoid organ karyocytes, T-, and B-lymphocytes in irradiated animals. IM862 demonstrated a normalizing action to a great degree on T-system immunity. IM862 limited the increase in the percent of leukocyte migration at 5 days after irradiation and normalized this index by 20 days after radiation exposure. The cation protein count in the blood neutrophils was also normalized at 10 and 20 days after irradiation.

These results show that administration of IM862 had a positive effect on post-radiation lymphocyte restoration generally and within subpopulations of lymphoid organs of irradiated animals. IM862 also normalized the functional activity of the lymphocytes and neutrophils in the blood. Therefore, IM862 may restore the functional activity of the immune system following ionizing radiation.

### Example 106

This example demonstrates the efficacy of IM862 in the treatment of Dengue Fever. Administration of IM862 significantly shortened the duration of symptoms.

Forty-nine male patients (20-30 years old) participated in a study of the efficacy of IM862 in the treatment of Hemorrhagic Dengue Fever (HDF). All those examined were representatives of organized collectives and had the same type of working and living conditions prior to the onset of illness. The patients were referred to in-patient treatment to the 175th Military Hospital in Ho Chi Minh City during an epidemic of Dengue fever in southern Socialist Republic of Vietnam.

Diagnoses of HDF were established by criteria recommended by the WHO (1986). Serological analyses were performed at the Arbovirus Infections Laboratory of the Pasteur Institute (Ho Chi Minh City). Disease severity was 1-2 in all patients.

Clinical manifestations of HDF generally had the following characteristic traits. All fevers had a single wave character. The disease began acutely, with an elevation of body temperature, chills, headache (98.3%), dizziness (95%), and weakness (100%). Later symptoms included anorexia (95%), sleep disturbances (90%), xerostomia (85%), ophthalmalgia(58.6%) , scleral injection (55.2%), constipation (48.3%), cough (35%), photophobia (32.2%), lacrimation (24.1%), nausea (15%), vomiting (11.7%). Muffled heart tones were often present (86.7%). A functional systolic murmur was present at the apex in some patients (5.7%).

Many patients experienced myalgias and arthralgias during the acute phase of the illness. Exanthema and lymphadenopathy were present in only a portion of the patients (40.7% and 46.7%, respectively). Hepatomegaly and splenomegaly were present in most patients. Leukopenia, thrombocytopenia, and coagulopathies were common.

The patients were divided into one group treated with IM862 and one group not receiving IM862. IM862 was administered during the acute period of the illness to febrile patients to a total dose of 500 μg. The efficacy of therapy was determined by observation of the duration of the most frequently encountered signs and symptoms of the disease (Table 14).

TABLE 14
Basic Symptoms of Hemorrhagic Dengue Fever
during IM862 Administration to Patients.

Duration (days) of Symptoms

| | | No IM862 | |
|---|---|---|---|
| Index | IM862 administered | (control) | P |
| Fever | | | |
| Prescription 2- | 5.2 ± 0.3 | 7.3 ± 0.6 | <0.01 |
| 6 days | | | |
| Prescription 2- | 4.7 ± 0.2 | 7.3 ± 0.6 | <0.001 |
| 4 days | | | |
| Headaches | 8.1 ± 0.9 | 12.2 ± 1.1 | <0.05 |
| Weakness/fatigue | 9.3 ± 0.8 | 11.6 ± 1.2 | >0.05 |
| Anorexia | 7.5 ± 0.8 | 11.4 ± 1.1 | <0.01 |
| Insomnia | 7.1 ± 0.7 | 11.0 ± 1.3 | <0.05 |
| Hepatomegaly | 11.3 ± 0.8 | 15.8 ± 0.6 | <0.001 |
| Splenomegaly | 9.6 ± 0.7 | 13.3 ± 0.7 | <0.01 |
| Lumbar pain | 8.7 ± 0.7 | 13.8 ± 1.4 | <0.01 |
| Joint pain | 7.4 ± 0.7 | 12.2 ± 1.5 | <0.01 |
| Low back pain | 8.1 ± 0.8 | 12.7 ± 1.3 | <0.05 |
| Abdominal pain | 7.7 ± 1.1 | 11.2 ± 1.7 | >0.05 |
| Ophthalmalgia | 6.6 ± 0.8 | 11.0 ± 1.1 | <0.01 |

Administration of IM862 acceleration resolution of signs and symptoms of HDF.

### Example 107

This example demonstrates use of IM862 to treat sinusitis in a pediatric population by the methods of the present invention. The treatment was effective in curing the disease in many patients.

Patients were given combined therapy, including the introduction of antibiotics, desensitizing remedies, and physical therapy procedures. IM862 was introduced as a 0.01% solution to the affected maxillary sinuses through hospital drainage tubing in a dose of 20-50 g, depending on the child's weight, for 5-10 days.

The effectiveness of IM862 use was evaluate by clinical and laboratory testing. An immunological investigation was conducted before the prescription of IM862 and again upon completion of the therapy.

The group investigated included 51 children, 30 boys and 21 girls. There were 9 children between the ages of 4 and 6, 18 from 7-8, three children from 9-11, and 21 children from 12-14 years old.

All the patients were divided into two groups: the first group included 9 children with acute and subacute purulent maxillary sinusitis; the second group consisted of 42 children with aggravated chronic purulent maxillary sinusitis and Highmore ethmoiditis.

The length of illness in the first group was 1 to 4 days for two children, 5-15 days for 4 children, 16-30 days for 2, and more than a month for 1 child. In the cases of chronic maxillary sinusitis, the length of illness in 27 children was from 6 months to a year, in 9 from a year to three years, and in 6—more than three years. The duration of the aggravation in the second group was, in 21 children, from 5 to 15 days, in 18 children—from 16 days to 1 month, and in 3 patients—more than a month. In 7 children the infection was unilateral; in 44 bilateral infection was present.

Four patients with acute purulent maxillary sinusitis received treatment in the hospital for 3-5 days before hospitalization. This included antibiotic therapy and the use of physical therapy procedures. Two patients from the first group had received puncture of the affected maxillary sinuses along with irrigation and the subsequent introduction of antibiotic solutions in a 0,1% solution of levomisol. The treatment, however, was not effective.

Of 42 patients in the second group, 27 had received treatment for 1-2 weeks that included the administration of antibiotics into the nasal sinuses by the displacement method. They were also given physical therapy procedures (UHF, microwave therapy, electrophoresis of medicinal substances endonasally). No effect from this treatment was noted.

Patients of the first group presented with complaints of weakness, fatigue, and a subfebrile condition for the space of 5-15 days (in 6 patients), headaches, difficulty in nasal breathing, and a purulent nasal discharge. On objective examination, acute hyperemia and swelling of the nasal cavity mucosa were noted with purulent exudate. In 5 patients the course of the acute maxillary sinusitis was complicated by the presence of chromic adenoiditis. Radiography of the accessory nasal sinuses revealed hypolucency of the affected sinuses. Puncture of the maxillary sinuses produced a purulent exudate in all the patients. Crystallography of the pathological exudate revealed an elevation in the number of crystallization centers (up to 300-400) and a coarse design character with a large quantity of crossings and interlacings. Upon analyzing the immunological examination indices in children of the first group, shifts were revealed in the cellular immunity system—a sharp drop in the absolute and relative number of T-lymphocytes and a rise in the number of B-lymphocytes. Alterations in the system of humoral immunity were less pronounced—the concentration of the serum immunoglobulins A and M increased in 8 patients; the concentration of immunoglobulin G was within normal adult ranges. The quantity of circulating immune complexes exceeded the norm by more than two times.

Immune system indices of patients in the first group before treatment are cited in Table 1.

In patients of the second clinical group with chronic sinusitis, a less vivid clinical picture was noted, predominated by symptoms of extended intoxication—fatigue (in 35 patients), loss of appetite, irritability. Thirty-seven patients complained of headaches. Most had a mucopurulent (33 patients) or purulent (9 cases) nasal discharge. Stagnant hyperemia and cyanosis of the nasal cavity mucous membranes were determined rhinoscopically. Chronic adenoiditis was noted in 20 patients.

Radiography of the sinuses exhibited a decrease in transparency in the region of the maxillary and cribrate sinuses was discovered in 38 patients; in 12 there was a thickening of the sinus wall mucosa. Crystallography of the pathologic secretion was characterized by the presence of a great number of crystallization centers (180-300), a dense net of coarse crystal rays, and the presence of interlacings and crossings.

On immunological examination of patients from the second group, a reduction was seen in the absolute and relative quantity of T-lymphocytes and an elevation in the content of B-lymphocytes. The concentration of serum immunoglobulin A was higher than the adult norm in 10 patients. The immunoglobulin G concentration was higher than the adult norm in 6 patients, and that of immunoglobulin M was higher than the adult norm in 33 patients. The quantity of circulating immune complexes exceeded normal limits and correlated with the severity of the process. The findings on the state of the immune status of the second group of patients are cited in Table 15.

TABLE 15
Immune System Indices for Patients With Paranasal
Sinusitis Before Treatment With IM862.

| | Group 1 | | Group 2 | |
|---|---|---|---|---|
| | (acute and subacute) | | (chronic) | |
| Indices | % | Absolute | % | Absolute |
| Leukocytes | | 7700.0 ± 662 | | 5707.1 ± 251 |
| Lymphocytes | 36.0 ± | 2864.6 ± 296 | 35.2 ± 1.9 | 1993.5 ± 120 |
| | 0.6 | | | |
| T- | 33.7 ± | 985.0 ± 86.6 | 35.9 ± 0.8 | 727.5 ± 51.5 |
| lymphocytes | 0.9 | | | |
| B- | 21.3 ± | 635.3 ± 107.1 | 20.6 ± 0.9 | 403.6 ± 28.0 |
| lymphocytes | 1.7 | | | |
| O- | 44.0 ± | 1254.3 ± 123 | 42.6 ± 1.7 | 848.3 ± 59,7 |
| lymphocytes | 0.8 | | | |

| | | |
|---|---|---|
| Immune | 210.0 ± 27.5 | 113.8 ± 10.3 |
| complexes | | |
| Lysozyme | 26.6 ± 4.1 | 35.3 ± 5.8 |

During therapy in the first group of patients, an improvement was noted in 2-3 days. The quantity of purulent nasal discharge decreased, mucosal swelling in the nasal cavity disappeared, the patient's general sense of well-being, sleep, and appetite all improved. After 4-5 days of treatment, the exudate irrigated from the affected maxillary sinuses decreased in volume and attained a mucous character and the quantity of it decreased.

Cessation of exudation (pure fluid upon maxillary irrigation) was noted on the 6th day of treatment in 5 patients, and in 7-9 days of treatment in 3 patients with acute purulent maxillary sinusitis.

Crystallography of irrigated fluid at the end of treatment was characterized by a decrease in the quantity of crystallization centers (30-50). Crystal ray crossings disappeared.

Repeat immunological examination following treatment revealed a statistically significant increase in the absolute and relative number of lymphocytes and T-lymphocytes. The B-lymphocyte content remained elevated. Also noted were a statistically significant decrease in the content of immune complexes and an increase in the quantity of lysozyme in the outwash from the maxillary sinuses.

The concentration of class M serum immunoglobulins remained elevated in 6 patients, and immunoglobulin A remained elevated in 2 patients. The patients immune status in cases of acute purulent maxillary sinusitis after treatment is reflected in Table 16.

During the process of treating patients with chronic purulent maxillary sinusitis and Highmore ethmoiditis using IM862, a gradual improvement in condition was noted. At 3-4 days of treatment an increase in exudation from the affected sinuses of 10-15% was registered as compared with the second 24-hour period, which was a favorable prognostic sign.

Following 4-5 days of treatment, rhinoscopy demonstrated disappearance of edema of the nasal mucosa in 32 patients. Nasal breathing and the general sense of wellness normalized in 38 patients. The exudate irrigated from the sinuses attained a mucous character in 3-4 days in 27 patients; in 5-6 days in 13; and in 7-9 days in two patients. Cessation of exudation was noted in 18 patients in 4-6 days; in 20 in 7-9 days; and in 4 in 10-14 days of treatment.

Control crystallograms of the irrigated fluid were characterized by a decrease in the quantity of crystallization centers (50-60), by a delicate design, and by a lack of interlacings and crossings in 38 patients.

In two patients with Highmore ethmoiditis, the illness was more persistent. The exudation from the sinuses maintained itself for a span of 14 days in one case, and for 16 days in the other. One-and-a-half to two months after discharge, scant mucous discharge and nasal stuffiness remained in both patients.

After IM862 treatment in the second group of patients, changes were noted in the immunogram consisting of an increase in the number of lymphocytes, the absolute and relative numbers of T-lymphocytes, and the absolute number of B-lymphocytes. The quantity of circulating immune complexes decreased, while the lysozyme content in the outwash from the sinuses grew. The levels of the A and G serum immunoglobulins, elevated before the start of treatment, normalized in 10 patients and 6 patients, respectively. The concentration of immunoglobulin M remained elevated in 24 patients.

Changes in the immune status of patients in the second group after treatment are reflected in Table 3 below. Table 16. The Patients Immune Status in Cases of Acute Purulent Maxillary Sinusitis After Treatment With IM862.

| | Before | After | | |
|---|---|---|---|---|
| | Treatment | Treatment | | |
| Indices | c IM862 | c IM862 | t | p |
| Leukocytes abs. | 7700.0 ± 662.1 | 7200.0 ± 234.5 | 0.7 | >0.05 |
| Lymphocytes % | 36.0 ± 0.6 | 44.7 ± 1.7 | 4.9 | <0.001 |
| abs. | 2864.6 ± 296.8 | 3281.3 ± 235.0 | 1.1 | >0.05 |
| T-lymphocytes % | 33.7 ± 0.9 | 41.8 ± 1.2 | 5.3 | <0.001 |
| abs. | 985.0 ± 86.6 | 1420.3 ± 131.6 | 2.8 | <0.05 |
| B-lymphocytes % | 21.3 ± 1.7 | 21.9 ± 2.1 | 0.2 | >0.05 |
| abs. | 635.3 ± 107.1 | 778.0 ± 89.9 | 1.0 | >0.05 |
| O-lymphocytes % | 44.0 ± 0.8 | 34.3 ± 2.4 | 3.8 | <0.005 |
| abs. | 1254.3 ± 123.5 | 1082.0 ± 21.0 | 1.4 | >0.05 |
| Immune complexes | 210.0 ± 27.5 | 113.3 ± 15.3 | 3.1 | <0.01 |
| Lysozyme | 26.6 ± 4.1 | 45.0 ± 5.9 | 2.6 | <0.05 |

note:
IM862-0.01% solution to the affected maxillary sinuses through hospital drainage tubing in a dose of 20-50 g, depending on the child's weight, for 5-10 days.

TABLE 17
Changes in the Immune Status of Patients with Chronic Purulent
Maxillary Sinusitis and Highmore Ethmoiditis After Treatment.

| | Before | After | | |
|---|---|---|---|---|
| | Treatment | Treatment | | |
| Indices | c IM862 | c IM862 | t | p |
| Leukocytes abs. | 5707.1 ± 251.0 | 6650.0 ± 262.2 | 2.6 | >0.05 |
| Lymphocytes % | 35.2 ± 1.3 | 41.5 ± 2.6 | 1.95 | <0.05 |
| abs. | 1993.5 ± 120.6 | 2761.8 ± 212.6 | 3.1 | <0.01 |
| T-lymphocytes % | 35.9 ± 0.8 | 41.8 ± 1.0 | 4.6 | <0.001 |
| abs. | 727.5 ± 51.5 | 1109.8 ± 74.1 | 4.2 | <0.001 |
| B-lymphocytes % | 403.6 ± 28.0 | 20.7 ± 0.7 | 0.1 | >0.05 |
| abs. | | 562.0 ± 38.1 | 3.3 | >0.05 |
| O-lymphocytes % | 42.6 ± 1.7 | 37.4 ± 1.9 | 2.1 | <0.05 |
| abs. | 848.3 ± 59.7 | 1052.5 ± 109.3 | 1.6 | >0.05 |
| Immune complexes | 113.8 ± 10.3 | 90.8 ± 10.0 | 1.6 | <0.05 |
| Lysozyme | 35.3 ± 5.8 | 52.4 ± 4.1 | 2.4 | <0.05 |

note:
the second group consisted of 42 children with aggravated chronic purulent maxillary sinusitis and Highmore ethmoiditis

The introduction of IM862 into the affected maxillary sinuses was accomplished through hospital drainage tubing. The sinus was preliminarily irrigated with a 0.9%. solution of sodium chloride, then IM862 was introduced in the form of a 0.01% solution in a dose of 20-50 μg, depending on the weight of the child (if both maxillary sinuses were involved, the daily IM862 dose was cut in half). During the time that the exudation had a purulent character, IM862 was introduced along with 2 ml of a 0.5% solution of dioxydine. During the presence of a mucous exudate in the sinus, IM862 alone was introduced—saline solution was used as a solvent in the proportion of 1:5 (for example: 0.2 ml of a 0.01% IM862 solution and 1 ml of a 0.9% solution of sodium chloride).

Among the first group of patients, IM862 was used for a course of 4-5 days in 5 cases, and 6-7 days in 4 cases. In the second group, the duration of IM862 use was 4-5 days in 24 patients, 6-7 days in 15, and 8-10 days in 3.

The average hospital stay in the first group was 9.2 days, in the second—8 days.

The therapeutic action of IM862 during localized use for treating various forms of paranasal sinusitis was evidenced by normalization of nasal breathing, decrease in nasal cavity mucosal edema, cessation of exudation from the involved sinuses, and normalization of the general sense of wellness and the immune status indices.

### Example 108

This example demonstrates use of IM862 for the treatment of Psoriasis. Patients demonstrated improvement in clinical status and normalization of immunological parameters during treatment.

A comparative study of immunological, coagulation, and acute phase protein parameters was conducted on 30 patients with disseminated forms of psoriasis on normal treatment. Twenty-seven patients had widespread lesions of the smooth skin and scalp; two had psoriatic erythroderma. The period of illness in the majority of those examined exceeded 5 years. In two persons the nail plates were involved in the form of a “thimble” symptom.

Twenty-nine patients noted that a worsening of the pathologic process occurred during the winter. Subjective sensations were generally absent, however, in 6 an insignificant periodic pruritus was noted. The patients with erythroderma complained of chills and a feeling of skin tightening. The following were accompanying illnesses: dental caries—8, chronic tonsillitis—5, neurasthenia—2, atherosclerosis of the cerebral vessels—2 persons.

Basic clinical symptoms in those examined from this group are presented in Table 18.

TABLE 18
Dynamic of Basic Clinical Symptoms
in Patients With Psoriasis (Days).

| | | Smooth skin | |
|---|---|---|---|
| Indices | Smooth Skin | and scalp | Erythroderma |
| Pale | | 7. ± 0.4 | 9.9 ± 0.41 |
| effluorescence | | | |
| Flattening of | 7. ± 0.9 | 9.7 ± 0.35 | 19. ± 0.47 |
| erupted | | | |
| elements | | | |
| Beginning of | 21. ± 1.72 | 21.8 ± 1.72 | 30.7 ± 1.44 |
| erupted | | | |
| element | | | |
| disappearance | | | |
| Period of | | | 24.7 ± 0.2 |
| leukocyte | | | |
| count | | | |
| normalization | | | |
| Period of ESR | | | 30 ± 1.4 |
| normalization | | | |
| Period of | | | 14 ± 1.3 |
| temperature | | | |
| normalization | | | |
| Hospital stay | 30.1 ± 1.3 | 32.3 ± 1.72 | 49.2 ± 1.44 |
| (in days) | | | |

A leukocyte count greater than 18×10⁹/1 and an ESR elevation higher than 30 mm/hr. were recorded only during psoriatic erythroderma. In other forms, an insignificant leukocytosis as high as 9×10⁹/1 and a normal ESR were revealed.

Patients were treated under identical conditions and received therapy of vitamins B₁₂, B6, and A, folic acid, pyrogens, sedatives, and desensitizing preparations. Salidol ointment and 3-5% sulfur-salicylic ointments were used locally.

Basic immunological and coagulation assays were conducted on all patients. The findings obtained were compared with the results of investigations done on healthy people between the ages of 18-40 years. Our observations showed (Table 19) that in patients with psoriasis there is an increase in the lymphocyte count. At the same time, a drop is noted in the quantity of T-lymphocytes, a decrease in the level of T-helpers and suppressors, and a growth in the concentrations of IgA, M, and to a lesser degree, IgG.

In the reconvalescence period toward the end of treatment, the lymphocyte count in patients decreased and the T-lymphocyte number was unchanged. The content of T-active and B-lymphocytes even showed a decrease. The number of T-helpers and suppressors, and the immunoglobulin concentration as well, remained at the previous level.

TABLE 19
Indices of Cellular and Humoral Immunity in
Patients with Widespread Forms of Psoriasis.

| | | Control | |
|---|---|---|---|
| | | Before | After |
| | Healthy | treatment | treatment |
| Indices | p = 40 | p = 30 | p = 30 |
| Leukocytes 10⁹/l | 5.8 ± 0.1 | 6.6 ± 0.1 | 6.7 ± 0.1 |
| Lymphocytes | 1.74 ± 0.12 | 1.90 ± 0.06 | 1.68 ± 0.03 |
| 10⁹/l | | | p₂ 0.001 |
| T-lymphocytes | 0.89 ± 0.66 | 0.547 ± 0.04 | 0.503 ± 0.012 |
| 10⁹/l | | | |
| T-active | 0.51 ± 0.013 | 0.426 ± 0.025 | 0.342 ± 0.017 |
| lymphocytes | | | |
| 10⁹/l | | | |
| B-lymphocytes | 0.462 ± 0.02 | 0.356 ± 0.014 | 0.322 ± 0.016 |
| 10⁹/l | | | |
| T-helpers 10⁹/l | 0.606 ± 0.04 | 0.284 ± 0.02 | 0.269 ± 0.01 |
| T-suppressors | 0.284 ± 0.016 | 0.263 ± 0.015 | 0.234 ± 0.008 |
| 10⁹/l | | | |
| Co-relationship | 2.1 | 1.1 | 1.1 |
| Tx:Tc | | | |
| IgG ME/ml | 134 ± 5.6 | 143 ± 7.3 | 146 ± 4.4 |
| IgA ME/ml | 98.3 ± 6.2 | 140 ± 5.1 | 159 ± 5.1 |
| IgM ME/ml | 127 ± 9.7 | 153 ± 5.9 | 179 ± 4.0 |

### Example 109

This example demonstrates the efficacy of treatment of Staphylococcal skin diseases with IM862. Use of IM862 enhanced the efficacy of treatment with a variety of antibiotics.

IM862 was used in the form of a 0.01% solution (100 μg of the preparation in one IV tube). The preparation was administered intranasally, daily, at 100 μg for a course of 5 days.

The effectiveness of IM862 use was evaluated according to the subjective impression of the patient, clinical findings, body temperature, general clinical and immunological testing of the peripheral blood. Also, bacteriological investigation of the disease agents were conducted before and after treatment with IM862.

IM862 was administered to 59 chronic pyoderma patients. In 52, its influence on the antibiotic resistance of the staphylococcal foci of the skin lesions was studied. The patients ranged in age from 18 to 56 years old. Thirty-two men and 27 women were studied. Duration of illness was from 5 months to 16 years. Of these, 36 had been treated repeatedly with standard methods without significant clinical effect (a lack of recovery or insignificant clinical improvement). Clinical syndromes of the patients are presented in Table 20 below.

TABLE 20
Distribution of Patients Receiving IM862, by Contingents.

| | Number of Patients | | | | | | |
|---|---|---|---|---|---|---|---|
| | | Men | | | | Women | |
| Nosological Form | In All | 18-30 yrs | 31-43 yrs | 44-56 yrs | 18-30 yrs | 31-43 yrs | 44-56 yrs |
| Chronic recurring | 3 | 1 | — | — | 1 | 1 | — |
| osteal folliculitis | | | | | | | |
| Vulgar Sycosis | 1 | — | 1 | — | — | — | — |
| Comedones | 19 | 12 | — | — | 6 | 1 | — |
| papulosa-pustulosa | | | | | | | |
| Chronic recurring | 1 | — | — | — | — | 1 | — |
| folliculites deep | | | | | | | |
| Chronic furunculosis | 11 | 1 | 2 | 1 | 4 | 1 | 2 |
| Abscessing and | 14 | 7 | — | — | 6 | 1 | — |
| indurative comedones | | | | | | | |
| Chronic recurring | 1 | 1 | — | — | — | — | — |
| hydradenitis | | | | | | | |
| Chronic abscessing | 7 | 4 | 2 | — | — | — | 1 |
| pyoderma | | | | | | | |
| Chronic ulcerative | 2 | — | — | — | — | 2 | — |
| pyoderma | | | | | | | |
| Total | 59 | 26 | 5 | 1 | 17 | 7 | 3 |

For determining the norm of T- and B-system immunity and non-specific resistance, 175 healthy donors (men & women), aged 18 to 50 years, were examined. Lymphocytes were extracted from the heparinized (25 U/ml) peripheral blood of healthy and ill persons in a ficoll-urotrast density gradient. T-lymphocytes were identified by spontaneous rosette-formation with ram erythrocytes. The T-lymphocyte subpopulations were determined with the aid of mouse monoclonal antibodies, OKT4 and OKT8, as well as with the aid of a theophylline resistance test. B-lymphocytes were determined by rosette-formation with mouse erythrocytes. The B-lymphocyte subpopulations were determined according to the presence of various classes of immunoglobulins (Ig+, IgM⁺, IgG⁺, IgA⁺) on their surfaces with the aid of monospecific antibodies. In serum, the content of the immunoglobulins M, G, & A, and the C₃-component of complement was determined by using monospecific sera to human immunoglobulins with the radial immunodiffusion method. The percentage of migration inhibition in a LMIR with PHA and ConA was determined using the hemolytic staphylococcus allergen (HSA). The phagocytic activity of the neutrophils (percent and absolute quantity of phagocytic polymorphonuclear leukocytes) and the phagocytic index (mean quantity of microorganisms absorbed by a single phagocyte) were studied with the use of a museum culture of the “Oxford 209P” strain. Other analyzed parameters were the cationic protein content (MCC) in the neutrophils, the serum immune complexes (CIC), the natural killer activity (CI) by the 3H-uridine test, and the quantity of neutrophils forming rosettes with sheep erythrocytes.

The antibiotic-sensitivity of the microorganisms was determined by the double-series cultivation method using the MIC-2000 automated system and 15 antibiotics for testing.

The results of the clinical, immunological, and bacteriological investigations were subject to a statistical work-up using the parametric method of statistical analysis (Student's t-test).

Clinical treatment using IM862 provided resolution of dermal infection foci, and curtailed (towards the end of the course) the appearance of fresh elements. During this time the preparation was well-tolerated. No side effects from its use were noted in any of the patients. A clinical analysis of the treatment (examination) conducted is presented in Table 21, where statistically reliable changes after IM862 use were not revealed.

TABLE 21
Indices of General Clinical Testing in Patients
with Chronic Pyoderma Who Receive IM862.

| | Before | After treatment | |
|---|---|---|---|
| Index | treatment | with IM862 | p |
| Body temperature | 36.4 ± 0.1 | 36.2 ± 0.1 | >0.05 |
| (morning) | | | |
| ESP, mm/hr | 13.2 ± 1.3 | 11.2 ± 1.4 | >0.05 |
| Erythrocytes ×10¹²/l | 5.4 ± 0.7 | 5.2 ± 0.6 | >0.05 |
| Leukocytes ×10⁹/l | 7.89 ± 0.36^{x} | 7.75 ± 0.36 | >0.05 |
| Neutrophils % | 61.1 ± 1.5 | 59.8 ± 1.6 | >0.05 |
| Bacilli/nuclei % | 5.8 ± 0.6 | 3.8 ± 0.6 | >0.05 |
| Segm./nuclei % | 59.4 ± 1.7 | 61.9 ± 1.4 | >0.05 |
| Monocytes % | 6.1 ± 0.4 | 5.7 ± 0.4 | >0.05 |
| Basophils % | 0.1 ± 0.1 | 0.1 ± 0.1 | >0.05 |

x-p<0.05-statistically relevant as compared to indices of healthy (normal) persons.

Immunological analyses of patients revealed a decrease in the quantity of T-lymphocytes (E-RFC) (p<0.05), the relative and absolute quantity of T-helpers (OKT4+) (p<0.05), the absolute quantity of Eₜᵣ-RFC (p<0.05), and the T4/T8 ratio (p<0.05), and an elevation in the percentage of migration inhibition in a LMIR with PHA (p<0.01), Con A (p<0.001), and HSA (p<0.05). After IM862 treatment, the relative and absolute quantity of T-lymphocytes (E-RFC) (p<0.05), the relative and absolute number of T-helpers (Eₜᵣ-RFC) (p<0.05), the absolute quantity of OKT4+(p<0.05), and the T4/T8 coefficient (p<0.05) all increased. The percentage of migration inhibition in a-LMIR with PHA (p<0.05), Con A (p<0.01), and HSA (p<0.01) decreased. These parameters demonstrate the immunomodulating influence of IM862 by the normalization of cellular immunity indices.

Analysis of indices of humoral immunity (Table 23) revealed a decrease in the absolute quantity of B-lymphocytes (Em-RFC) (p<0.01) and an increase in the relative and absolute quantity of B-cell subpopulations—B_{Ig}⁺ (p<0.001) and the content of class G & A (p<0.01) serum immunoglobulins. After treatment, the absolute quantity of B_{Ig}⁺-lymphocytes (p<0.05) was reduced, thus indicating IM862's normalizing influence on the B-lymphocytes subpopulation ratios.

Analysis of the indices of nonspecific resistance (Table 24) revealed a decrease in the relative quantity of rosette-forming neutrophils (Eₙ-RFC) (p<0.05), the percent and absolute quantity of neutrophilic phagocytes (p<0.01), the phagocytic index (p<0.05), the content of nonenzymatic cationic proteins in the neutrophils (MCC) (p<0.01), the level of the C₃-component of complement in the serum, and the activity of the natural killers (CI) (p<0.01). IM862 strengthened the processes of phagocytosis and normalized the state of natural killer activity.

TABLE 22
Cellular Immunity Indices in Patients
With Chronic Pyoderma Receiving IM862.

| | | Chronic Pyoderma | |
|---|---|---|---|
| Indices | Healthy | Before IM862 | After IM862 |
| Leukocytes | 6.71 ± 0.17 | 7.89 ± 0.36^{xx} | 7.75 ± 0.33 |
| ×10⁹/l | | | |
| Lymphocytes % | 28.0 ± 0.6 | 29.4 ± 1.3 | 30.8 ± 1.4 |
| ×10⁹/l | 2.01 ± 0.09 | 2.26 ± 0.12 | 2.41 ± 0.14 |
| T-lymphocytes % | 61.4 ± 1.6 | 62.8 ± 1.9 | 69.3 ± 1.9^{y} |
| E-RFC ×10⁹/l | 1.70 ± 0.12 | 1.38 | 1.68 ± 0.12^{y} |
| T-helpers % | 35.3 ± 2.7 | 13.7 ± 1.6^{x} | 20.3 ± 2.9 |
| OKT4⁺ ×10⁹/l | 0.65 ± 0.05 | 0.30 ± 0.03^{x} | 0.46 ± 0.05^{y} |
| T-helpers % | 47.1 ± 2.0 | 43.2 ± 2.3 | 51.7 ± 2.8^{y} |
| Etr-RFC ×10⁹/l | 1.32 ± 0.1 | 1.0 ± 0.07^{x} | 1.28 ± 0.09^{y} |
| T-suppressors % | 21.3 ± 0.9 | 17.9 ± 1.4 | 17.1 ± 1.9 |
| OKT8 ×10⁹/l | 0.41 ± 0.03 | 0.37 ± 0.04 | 0.39 ± 0.04 |
| T-suppressors % | 14.3 ± 1.6 | 14.1 ± 1.8 | 17.9 ± 2.4 |
| Etr-RFC ×10⁹/l | 0.39 ± 0.05 | 0.34 ± 0.04 | 0.45 ± 0.08 |
| T4/T8 | 1.64 ± 0.12 | 0.2 ± 0.17^{x} | 1.32 ± 011^{y} |
| Etr-RFC/Ets-RFC | 3.29 ± 0.19 | .86 ± 3.12 | 5.32 ± 1.00 |
| LMIF with PHA % | 35.9 ± 2.6 | 50.2 ± 2.7^{xx} | 40.2 ± 3.9^{y} |
| LMIF with ConA % | 49.4 ± 3.3 | 76.3 ± 4.9^{xxx} | 58.3 ± 3.9^{yy} |
| LMIF with HSA % | 95.1 ± 5.7 | 114.5 ± 7.6^{x} | 84.9 ± 5.5^{yy} |

TABLE 23
Humoral Immunity Indices in Patients With
Chronic Pyoderma Receiving IM862.

| | | Chronic Pyoderma | |
|---|---|---|---|
| | | Before | After |
| | | Treatment | Treatment |
| Indices | Healthy | With IM862 | With IM862 |
| B-lymphocytes % | 18.1 ± | 13.5 ± 1.4 | 12.1 ± 1.5 |
| Em-RFC ×10⁹/l | 1.4 | 0.30 ± 0.04^{xx} | 0.28 ± 0.04 |
| | 0.49 ± | | |
| | 0.04 | | |
| B-lymphocytes % | 13.8 ± | 16.1 ± 2.2 | 16.9 ± 1.7 |
| (Ig⁺) ×10⁹/l | 1.2 | 0.40 ± 0.07 | 0.34 ± 0.05 |
| | 0.29 ± | | |
| | 0.02 | | |
| B-lymphocytes % | 6.4 ± | 4.4 ± 0.8 | 4.4 ± 0.6 |
| (IgM⁺) ×10⁹/l | 0.7 | 0.11 ± 0.02 | 0.10 ± 0.01 |
| | 0.12 ± | | |
| | 0.01 | | |
| B-lymphocytes % | 4.1 ± | 11.4 ± 2.01^{xxx} | 14.7 ± 2.16 |
| (IgG⁺) ×10⁹/l | 0.05 | 0.37 ± 0.015^{xxx} | 0.19 ± 0.023^{y} |
| | 0.08 ± | | |
| | 0.008 | | |
| B-lymphocytes % | 2.2 ± | 2.4 ± 0.4 | 2.4 ± 0.3 |
| (IgA⁺) ×10⁹/l | 0.2 | 0.08 ± 0.021 | 0.06 ± 0.008 |
| | 0.04 ± | | |
| | 0.004 | | |
| IgM g/l | 1.15 ± | 1.38 ± 0.17 | 1.25 ± 0.12 |
| | 0.06 | | |
| IgG g/l | 11.5 ± | 14.1 ± 0.49^{xx} | 15.5 ± 0.73 |
| | 0.5 | | |
| IgA g/l | 1.90 ± | 2.38 ± 0.14^{xx} | 2.40 ± 0.18 |
| | 0.08 | | |

^{x}p < 0.05 statistically relevant as compared with healthy (normal) persons
^{xx}p < 0.01 statistically relevant as compared with healthy (normal) persons
^{xxx}p < 0.001 statistically relevant as compared with healthy (normal) persons
^{y}p < 0.05 statistically relevant as compared with analogous indices before IM862 use

TABLE 24
Non-Specific Resistance Indices in Patients
With Chronic Pyoderma Receiving IM862.

| | | Chronic Pyoderma | |
|---|---|---|---|
| Indices | Healthy | Before IM862 | After IM862 |
| Neutrophils % | 63.1 ± 1.1 | 61.1 ± 1.5 | 59.8 ± 1.6 |
| × 10⁹/l | 4.41 ± 0.18 | 4.98 ± 0.29 | 4.54 ± 0.25 |
| Neutrophils % | 17.2 ± 1.4 | 11.1 ± 1.1^{x} | 14.7 ± 1.6 |
| En-RFC × 10⁹/l | 0.60 ± 0.05 | 0.54 ± 0.08 | 0.7 ± 0.11 |
| Monocytes % | 7.0 ± 0.28 | 6.14 ± 0.40 | 5.65 ± 0.40 |
| × 10⁹/l | 0.55 ± 0.03 | 0.48 ± 0.03 | 0.43 ± 0.04 |
| Phagocytic | 60.0 ± 1.4 | 44.7 ± 2.2^{xx} | 48.9 ± 2.8 |
| activity* % | 3.15 ± 0.17 | 2.19 ± 0.19^{xx} | 2.36 ± 0.27 |
| × 10⁹/l | | | |
| Phagocytic | 4.92 ± 0.05 | 4.51 ± 0.18^{x} | 5.31 ± 0.20^{y} |
| index % | | | |
| × 10⁹/l | | | |
| Cathion | 1.61 ± 0.04 | 1.26 ± 0.04^{xx} | 1.46 ± 0.05^{y} |
| proteins | | | |
| MCC × 10⁹/l | | | |
| C3 complement | 0.84 ± 0.02 | 0.76 ± 0.01^{xx} | 0.770.02 |
| g/l | | | |
| NK-activity | 45.07 ± 2.82 | 29.86 ± 3.35^{x} | 45.18 ± 2.55^{y} |
| (CI) % | | | |
| CIC units | 44.0 ± 1.6 | 39.6 ± 3.6 | 45.9 ± 4.9 |

*phagocytosis with staph. cells
^{x}p < 0.05 statistically relevant as compared with healthy (normal) persons
^{xx}p < 0.01 statistically relevant as compared with healthy (normal) persons
^{y}p < 0.05 statistically relevant as compared with analogous indices before IM862 use

Staphylococci extracted from dermal lesions were initially sensitive to 10 of the 15 antibiotics being assayed (Table 25). After IM862 use, organisms from the infected foci were sensitive to 14 of the 15 tested antibiotics.

The clinical effectiveness of IM862 use in combination with antibiotics and other stimulatory agents was demonstrated in 36 patients having infections resistant to standard therapy. Patients were assigned to two categories, those in whom clinical recovery had not been achieved with standard therapy and those in whom recurrences had arisen less than 3 months after cessation of treatment. Table 24 shows that the inclusion of IM862 into the therapy of chronic pyoderma provided clinical recovery in 27 patients, considerable improvement in 8, and improvement in 1. All patients demonstrated some degree of improvement. Subsequent observation for 6 months after discharge showed that relapses occurred in only 3 patients, and were characterized by a mild course.

TABLE 25
Changes in Antibiotic-Sensitivity of the Microbial Populations
Receiving IM862 (method of series cultivation).

MIC g/ml

| Name of Antibiotic | Before IM862 | After IM862 |
|---|---|---|
| Penicillin | 9.45 ± 0.28^{x} | 2.52 ± 0.70^{yy} |
| Bicillin-3 | 9.24 ± 0.53^{x} | 2.42 ± 1.02^{yy} |
| Oxycillin | 5.11 ± 0.63^{x} | 1.56 ± 0.45^{yy} |
| Ampicillin | 8.10 ± 0.49^{x} | 2.64 ± 0.60^{yy} |
| Ampiox | 7.67 ± 0.70^{x} | 3.06 ± 0.3^{yy} |
| Levomicetin | 9.17 ± 0.36^{x} | 4.58 ± 0.67^{yy} |
| Streptomycin | 6.71 ± 0.59^{x} | 3.39 ± 0.66^{yy} |
| Monomycin | 6.34 ± 0.55^{x} | 2.01 ± 0.46^{yy} |
| Kanamycin | 6.32 ± 0.58^{x} | 2.09 ± 0.54^{yy} |
| Gentamycin | 5.47 ± 0.92^{x} | 1.32 ± 0.49^{yy} |
| Sizamycin | 3.46 ± 0.90 | 1.59 ± 0.58 |
| Cenorin | 5.69 ± 0. | 1.82 ± 0.67^{yy} |
| Cefamezin | | 1.33 ± 0.53^{yy} |
| Keflin | 4.49 ± 0.93 | 1. ± 0.69^{y} |
| Kefzol | 4.61 ± 0.87 | 1.86 ± 0.93^{y} |

^{x}index raising the boundary MIC value corresponding to sensitivity to the antibiotic
^{y}p < 0.05 statistically relevant as compared with analogous indices before IM862 use
^{yy}p < 0.0001 statistically relevant as compared with analogous indices before IM862 use

TABLE 26
Clinical Outcomes of the Complex Treatment of
Patients With Chronic Pyoderma Using IM862.

| | | Clinical Outcomes | | | |
|---|---|---|---|---|---|
| | | | | No | |
| Nosological | Recov- | Considerable | Improve- | Ef- | Wor- |
| Forms | ery | Improvement | ment | fect | sening |
| Chronic recurring | 3 | — | — | — | — |
| osteofolliculitis | | | | | |
| Chronic recurring | 1 | — | — | — | — |
| folliculitis-deep | | | | | |
| Papulous-pustulous | 4 | 3 | — | — | — |
| comedones | | | | | |
| Abscessing and | 4 | 1 | 1 | — | — |
| indurative | | | | | |
| comedones | | | | | |
| Chronic | 10 | — | — | — | — |
| furunculosis | | | | | |
| Chronic abscessing | 5 | 2 | — | — | — |
| pyoderma | | | | | |
| Chronic ulcerative | — | 2 | — | — | — |
| pyoderma | | | | | |
| Total number of | 27 | 8 | 1 | — | |
| patients | | | | | |

### Example 109

This example demonstrates the use of IM862 in the treatment of prostatitis.

A clinical study of the preparation was conducted in 34 chronic prostatitis patients having active inflammation. Patients were aged 22 to 45 years old. Diagnosis was established by patient complaints, a medical history, palpation of the prostate, and laboratory investigation methods (microscopic examination of prostate gland secretions, spermography, three-cup urinalysis). All patients had received repeated standard treatments (antibacterial therapy, uroantiseptics, spasmolytics, ganglion blockers, novocaine paraprostate and presacral blocks, prostate gland massage, physical procedures, therapeutic exercise, etc.) with consequent partial and short-term remissions.

The control group consisted of 14 chronic prostatitis patients in the active phase of inflammation between the ages of 23 and 45, also having undergone long-term treatment using traditional methods.

Prior to IM862 treatment, an immunological examination was conducted consisting of the following methods:

Tests of E- and EAS-rosette formations for determining T- and B-lymphocyte counts.

A determination of the serum immunoglobulin concentrations in the three basic classes.

A determination of the phagocytic activity of the leukocytes (neutrophils).

Titration of complement.

A determination of circulating immune complexes.

Intramuscular injections of IM862 were made at 100 μg/dose for 5 days (a 500 μg treatment course). A traditional treatment plan was followed simultaneously, using antibacterial preparations, uroantiseptics, physical procedures, prostate gland massage, and therapeutic exercise.

After completion of the treatment course, 10 days after the last IM862 injection, all the patients again received the above-stated combined immunological examination. Measurement reliability of the obtained indices was evaluated with the use of variable statistics methods.

The clinical effectiveness of the preparation was assessed by generally-accepted criteria for treatment effectiveness in patients with chronic prostatitis.

The persistent disappearance (over an observation period of 1-1.5 months) of painful sensations, leukocyturia, the lowering of the leukocyte count to normal levels in prostate gland secretions and ejaculate, and the overall improvement in the general state of the patient were all deemed to be a good result.

The normalization of all laboratory indices while complaints of pain persisted in the perineum, sacrum, anal region, scrotum, etc. was rated as a satisfactory result.

The lack, either during treatment or after its completion, of an improvement in clinical indices was considered to be an unsatisfactory result.

The patients comprising the control group underwent treatment by traditional means without the use of an immunomodulator. After the completion of the treatment course, these patients were given the same combined, clinical examination in the same time period, using the same recovery criteria already mentioned above.

A good result was noted in 26 patients (76.5%). After finishing the treatment course, the patients noticed significant improvement in their general condition and the disappearance of painful symptoms and dysuria. All of their laboratory indices returned to normal. The remaining 8 patients had a satisfactory result.

In the control group, 9 persons (64.3%) received a result of good, 3 patients (21.4%)—satisfactory, and 2 (14.3%)—unsatisfactory.

Immune system indices of patients receiving IM862, and in control group patients, studied before and after the treatment course, are cited in the tables below. The average values cited in Tables 27 and 28 of the indices before and after treatment remained within normal ranges for all patients, whereas individual differences could vary significantly.

To evaluate immune system impairments in chronic prostatitis patients, as well as to measure them after conducting a course of treatment with the immunomodulator, IM862, the patients were divided into three groups. The first group consisted of patients exhibiting normal initial indices. The second group was made up of patients with lowered initial indices, and the third had patients with elevated initial indices.

The data of Table 29 show that before initiating treatment, the T- and B-lymphocyte count in the majority of patients was normal (52.9% and 47.1%, respectively), or lowered (41.2% and 29.4%). Phagocytosis indices were raised in 76.8% of patients, lowered in 14.7% of patients, and remained within normal ranges in 8.8% of patients. A reduction in the liter of complement took place in a majority (41.2%) of patients. The quantity of circulating immune complexes was raised in 55.9% of patients.

Upon completion of treatment with IM862 use, a normalization was observed in all the elevated and lowered indices. Normal pre-treatment indices remained normal.

As illustrated in Table 30, the quantity of immunoglobulin A before treatment was normal in all patients. Immunoglobulin M was normal in the majority (97.1%) of patients and only in 2.9% of cases was elevated. The most pronounced changes are related to the indices of immunoglobulin G, the quantity of which was in normal ranges in 26 (76.5%) patients, elevated in 6 (17.7%) patients, and lowered in 2 (5.9%) patients. IM862 treatment showed a normalization in the indices of the immunoglobulins M and G in all three patient groups, where they had been measured before treatment.

In Tables 31 and 32, pre- and post-treatment immunological indices are reported in the control group demonstrating the pronounced immunomodulating effect of IM862 during treatment of chronic prostatitis patients. This conclusion was confirmed by clinical observations.

TABLE 27
Immunological Exam Indices Before and After IM862 Treatment.

| | | | After |
|---|---|---|---|
| | | | Treatment |
| | | Before | 100 μg i.m. |
| Index | Norm | Treatment | for 5 days |
| T-lymphocytes % | 40-70 | 45.76 ± 2.59 | 44.44 ± 2.34 |
| Abs. quantity ×10⁹ | | 0.91 ± 0.02 | 0.88 ± 0.07 |
| B-lymphocytes % | 30 | 24.29 ± 1.51 | 27.24 ± 1.44 |
| Abs. quantity ×10⁹ | | 0.48 ± 0.03 | 0.54 ± 0.10 |
| Phagocytosis % | 25 | 41.12 ± 3.76 | 39.41 ± 3.51 |
| Complement units | 29.5-31.0 | 29.46 ± 0.59 | 29.43 ± 0.53 |
| Imm. complexes | 0.06-0.08 | 0.086 ± 0.006 | 0.080 ± 0.004 |
| units | | | |
| IgA g/l | 0.8-5.2 | 2.50 ± 0.15 | 2.20 ± 0.12 |
| IgB g/l | 0.6-3.8 | 2.13 ± 0.17 | 2.12 ± 0.12 |
| IgG g/l | 6.0-18.0 | 12.59 ± 0.78 | 12.52 ± 0.63 |

TABLE 28
Immunological Exam Indices of Patients Not Receiving IM862.

| | | After Treatment |
|---|---|---|
| | | 100 μg i.m. |
| Index | Before Treatment | for 5 days |
| T-lymphocytes % | 43.43 ± 4.39 | 41.71 ± 5.77 |
| Abs. quantity ×10⁹ | 0.96 ± 0.01 | 0.92 ± 0.03 |
| B-lymphocytes % | 23.21 ± 2.33 | 24.79 ± 2.33 |
| Abs. quantity ×10⁹ | 0.46 ± 0.03 | 0.49 ± 0.08 |
| Phagocytosis % | 32.43 ± 2.63 | 29.71 ± 2.72 |
| Complement units | 29.23 ± 1.08 | 28.54 ± 1.08 |
| Imm. complexes | 0.093 ± 0.013 | 0.084 ± 0.006 |
| units | | |
| IgA g/l | 2.53 ± 0.26 | 2.39 ± 0.17 |
| IgB g/l | 2.13 ± 0.89 | 2.34 ± 4.11 |
| IgG g/l | 12.76 ± 1.21 | 12.53 ± 1.31 |

TABLE 29
Dynamic of Immunity indices Dependant on Initial Data
on Patients Receiving IM862. Immunity Indices

| | | | After |
|---|---|---|---|
| | | | Treatment |
| | | Before | 100 μg i.m. |
| Name | Level | Treatment | for 5 days |
| T-lymphocytes % | normal | 54.0 ± 2.05 | 50.0 ± 3.57 |
| | n = 18 | | |
| | lowered | 31.07 ± 1.03 | 37.9 ± 2.24 |
| | n = 14 | | |
| | elevated | 74.5 ± 2.12 | 40.0 ± 15.5 |
| | n = 2 | | |
| B-lymphocytes % | normal | 24.0 ± 0.59 | 29.2 ± 2.15 |
| | n = 16 | | |
| | lowered | 15.0 ± 1.22 | 26.0 ± 2.91^{x} |
| | n = 10 | | |
| | elevated | 36.5 ± 2.19 | 24.8 ± 2.91^{x} |
| | n = 8 | | |
| Phagocytosis % | normal | 23.33 ± 0.41 | 58.6 ± 17.98 |
| | n = 3 | | |
| | lowered | 16.64 ± 4.15 | 36.6 ± 8.54^{x} |
| | n = 5 | | |
| | elevated | 47.89 ± 3.99 | 37.7 ± 3.94 |
| | n = 26 | | |
| Complement | normal | 30.25 ± 0.17 | 29.3 ± 0.88 |
| units | n = 13 | | |
| | lowered | 26.34 ± 0.53 | 34.2 ± 0.93^{x} |
| | n = 14 | | |
| | elevated | 34.23 ± 0.86 | 30.7 ± 0.89 |
| | n = 7 | | |
| Imm. complexes | normal | 0.07 | 0.08 ± 0.006 |
| units | n = 7 | | |
| | lowered | 0.05 ± 0.001 | 0.07 ± 0.011 |
| | n = 8 | | |
| | elevated | 0.106 ± 0.008 | 0.08 ± 0.006^{x} |
| | n = 19 | | |

n - number of patients
^{x}p < 0.05

TABLE 30
Blood Immunoglobulins in Patients Receiving
IM862 Before and After Treatment.

| | | | After |
|---|---|---|---|
| | | | Treatment |
| | | Before | 100 μg i.m. |
| Indices | | treatment | for 5 days |
| Ig [symbol] l | normal in 34 | 2.50 ± 0.15 | 2.20 ± 0.12 |
| | patients | | |
| IgM g/l | normal in 33 | 2.01 ± 0.13 | 2.08 ± 0.12 |
| | patients | | |
| | elevated in 1 | 6.0 | 3.44 |
| | patient | | |
| IgG g/l | normal in 34 | 11.76 ± 0. 60 | 12.51 |
| | patients | | |
| | lowered in 2 | 3.70 ± 0.42 | 7.80 ± 1.13 |
| | patients | | |
| | elevated in 1 | 19.13 ± 0.31 | 14.71 ± 1.42 |
| | patient | | |

TABLE 31
Blood Immunoglobulins in Patients Receiving
IM862 Before and After Treatment.

| | | | After |
|---|---|---|---|
| | | | Treatment |
| | | Before | 100 μg i.m. |
| Indices | | treatment | for 5 days |
| Ig [symbol] l | normal in 14 | 2.53 ± 0.26 | 2.39 ± 0.17 |
| | patients | | |
| IgM g/l | normal in 1 | 1.93 ± 0.19 | 2.09 ± 0.24 |
| | patients | | |
| | lowered in 1 | 0.5 | 0.4 |
| | patients | | |
| | elevated in 1 | 6.2 | 7.4 |
| | patient | | |
| IgG g/l | normal in 11 | 12.56 ± 0.92 | 12.04 ± 0.98 |
| | patients | | |
| | lowered in 1 | 3.2 | 3.4 |
| | patients | | |
| | elevated in 2 | 18.6 ± 0.28 | 19.8 ± 0.28 |
| | patient | | |

TABLE 32
Dynamic of Immunity Indices Dependant on Initial Data
of Patients in the Control Group. Immunity Indices

| | | | After |
|---|---|---|---|
| | | | Treatment |
| | | Before | 100 μg i.m. |
| Name | Level | Treatment | for 5 days |
| T-lymphocytes % | normal | 51.29 ± 3.23 | 48.86 ± 6.79 |
| | n = 7 | | |
| | lowered | 28.83 ± 2.14 | 25.83 ± 1.31 |
| | n = 6 | | |
| | elevated | 76 | 87 |
| | n = 1 | | |
| B-lymphocytes % | normal | 24.0 ± 0.75 | 24.86 ± 3.43 |
| | n = 7 | | |
| | lowered | 14.50 ± 2.43 | 15.25 ± 2.38 |
| | n = 4 | | |
| | elevated | 33.07 ± 7.87 | 37.3 ± 5.49 |
| | n = 3 | | |
| Phagocytosis % | normal | 24.00 ± 1.41 | 27.34 ± 1.63 |
| | n = 2 | | |
| | lowered | 19.52 ± 2.12 | 12.00 ± 14.14 |
| | n = 2 | | |
| | elevated | 36.70 ± 2.49 | 33.33 ± 1.89 |
| | n = 10 | | |
| Complement | normal | 30.08 ± 0.41 | 27.34 ± 1.63 |
| units | n = 5 | | |
| | Lowered | 25.97 ± 1.17 | 26.75 ± 0.91 |
| | n = 6 | | |
| | elevated | 34.30 ± 2.02 | 34.10 ± 1.84 |
| | n = 3 | | |
| Imm. complexes | normal | 0.071 ± 0.007 | 0.081 ± 0.01 |
| units | n = 3 | | |
| | lowered | 0.049 ± 0.001 | 0.052 ± 0.002 |
| | n = 3 | | |
| | elevated | 0.12 ± 0.017 | 0.098 ± 0.006 |
| | n = 8 | | |

n - number of patients

### Example 110

This example demonstrates treatment of Mycobacterium tuberculosis with the methods of the present invention.

One hundred five patients having clinical forms of lung tuberculosis were observed and treated. Of these, 59 had an infiltrative process, 11 had a disseminated one, 10 had a fibrous/cavernous one, 9 had a cavernous one, and 16 had tuberculomas. All patients observed had a period of decline. Ninety-four patients were bacterial shedders. The patients were 19 to 60 years old and the group included 73 men and 32 women. In addition to standard testing, the following immunological indices were determined in the patients:

the quantity of T- and B-lymphocytes in spontaneous and complement rosette-formation reactions,

the IgA, IgM, and IgG content by the radial immunodiffusion method.

Complement activity by 100% hemolysis of sensitized sheep erythrocytes.

All the patients were divided into three observation groups:

Group 1—37 persons receiving, in addition to chemotherapy, IM862 at 50-100 μg on alternate days for 5 days.

Group 2—22 persons receiving, simultaneously with chemotherapy, decaris at 150 mg, 2 times per week, for 1.5-2 months.

Group 3—46 persons receiving chemotherapy without immunomodulators.

As a control for the laboratory tests, the immunological status of 37 healthy people was examined.

The efficacy of therapy was evaluated according to the following criteria: significant improvement, improvement, or no change.

For significant improvement, symptoms of intoxication completely disappeared, as did catarrhal phenomena, the greater part of infiltrates and foci resolved, the disintegration cavity closed up, and bacterial division ceased.

The category of improvement was understood to be the elimination of intoxication symptoms and rales in the lungs. There was a moderate resolution of infiltrates with partial consolidation of foci, and there was a decrease in the size of the caverns.

Following two months of treatment, significant improvement in the course of the tuberculous process along with enclosure of disintegration cavities was established in Group 1 (with IM862 use) in 9 persons; there was improvement in 27 persons. Within this same time period, in patients receiving decaris, significant improvement occurred in only one case, whereas there was clinical X-ray improvement in 18 patients. In 3 patients no improvement was noted. In Group 3 patients, the results were found to be analogous with those occurring in patients who received decaris.

After 4-6 months of in-hospital treatment, positive changes in the lungs of patients receiving IM862 occurred in 35 cases out of 37. One patient was discharged due to a breach of procedure. In one patient there was an onset of progression of the specific process which was linked to the principal medicinal resistance of the agent.

In Groups 2 and 3, disintegration cavities closed in 10 and 14 patients, respectively. That is, a combined therapy with IM862 use in the first stages of treatment was found to be more effective in comparison with decaris therapy and treatment without immunomodulators.

Analysis of immunological indices established that IM862 had a normalizing effect. IM862 lowered initially high immunity indices and elevated low ones. This was especially discernible after 2 months of observation. In patient groups receiving decaris or taking only chemopreparations, such regularities were not shown. IM862 was well-tolerated by the patients. Allergic reactions and other complications from its administration were not observed.

### Example 111

This example demonstrates the use of IM862 and antibiotics in the treatment of bacterial peritonitis. Mice having methicillin-resistant Staphylococcal aureus peritonitis treated with IM862 and antibiotics survived significantly longer than mice treated with antibiotics alone. The antibiotic tested was ampicillin that is typically ineffective against methicillin-resistant Staphylococcus.

Animals were inoculated intraperitoneally with 10×LD of Staphylococcal aureus suspended in brain-heart infusion broth containing 5 percent mucin. The antibiotic was administered s.c., i.p., or p.o., one hour following bacterial inoculation and deaths occurring during the subsequent three days are recorded.

Two control experiments were conducted. IM862 was administered as a pretreatment prior to microbe administration, and saline was administered i.p. in place of ampicillin. The number of survivors at 72 hours was determined. In the second control experiment, saline was administered as a pretreatment and 100 mg/kg ampicillin was administered i.p. during the hour following microbial inoculation. The number of survivors at 72 hours was again recorded. The influence of treatment with IM862 and ampicillin was determined as follows.

Test mice were divided into 3 groups. IM862 was administered as a pretreatment and ampicillin was administered i.p. in the first group at a dose of 100 mg/kg one hour following microbial inoculation. In the second group, the ampicillin was administered at 10 mg/kg i.p. In the third group, ampicillin was administered at a dose of 1 mg/kg i.p.

The effect of IM862 on survival in combination therapy with ampicillin was determined at two doses. In some mice, 0.01 mg/kg IM862 was administered for 3 days prior to bacterial microbial inoculation. In other mice, 0.001 mg/kg IM862 was administered for 3 days prior to microbial inoculation. The results are presented in Table 33 below.

TABLE 33
Antimicrobial Test Results (in vivo) with IM862 and
Ampicillin administration to mice subjected to intraperitoneal
S. Aureus (MR) 10 × LD.

| | | | Test | | |
|---|---|---|---|---|---|
| IM862 | Group | Ampicillin | animal | | Percent ip |
| 3 days | number | mg/kg 1 hr sc | number | Survivors | Survivors |
| Control | 1 | 100 | 5 | 0 | 0% |
| (0) | | | | | |
| 0.01 mg/kg | 2 | Control | 5 | 4 | 80 |
| | | (0) | | | |
| 0.01 mg/kg | 3 | 100 | 5 | 5 | 100 |
| 0.01 mg/kg | 4 | 10 | 5 | 5 | 100 |
| 0.01 mg/kg | 5 | 1 | 5 | 5 | 100 |
| 0.001 mg/kg | 6 | 100 | 5 | 5 | 100 |
| 0.001 mg/kg | 7 | 10 | 5 | 5 | 100 |
| 0.001 mg/kg | 8 | 1 | 5 | 5 | 100 |

Ampicillin MED > 100 mg/kg for S. Aureus (MR).
IM862 administered for 3 consecutive days prior to administration S. Aureus i.p.

There were no survivors in the control group of 5 test animals administered 100 mg/kg Ampicillin pre-treated with normal saline i.p. Four of 5 animals survived in the control group of animals that received 0.01 mg/kg IM862 3 days prior to bacterial inoculum and no antibiotic. No deaths occurred in animals receiving both IM862 and ampicillin.

### Example 112

This example demonstrates use of IM862 in the treatment of fungal infections. Local administration of IM862 resulted in less pronounced inflammation of the peritoneal cavity.

Two groups of guinea pigs were inoculated with Candida albicans by intraperitoneal injection. One group received intraperitoneal injection of IM862 at a dose of 1 μg/kg per day for four days following infection. The other group was not treated.

Animals were sacrificed at day 5 and day 10. The peritoneal cavities were examined for signs of inflammation. These signs included number of infectious foci adherent to the omentum, exudate, fibrin deposition, quantitative fungal cultures, and the number of neutrophils, macrophages, and lymphocytes infiltrating the peritoneum.

After 5 days, the inflammatory changes in the omentum did not principally differ between the groups. After 10 days, however, the inflammatory changes in the omentum of IM862 treated animals was less pronounced than in untreated animals. Quantitative fungal cultures revealed fewer fungi in the treated animals. Inflammatory infiltration of the omentum was also insignificant in the treated animals as compared to the untreated animals.

All publications, patents and patent applications mentioned in this specification are herein incorporated by reference into the specification to the same extent as if each individual publication, patent or patent application was specifically and individually indicated to be incorporated herein by reference.

Although the foregoing invention has been described in some detail by way of illustration and example for purposes of clarity of understanding, it will be obvious that certain changes and modifications may be practiced within the scope of the appended claims.

1. Use of a dipeptide having the formula X-Tryptophan or a pharmaceutically acceptable salt thereof, wherein X is glutamine, leucine or isoleucine in the manufacture of a medicament for any of the following:
(a) treatment of infection with a mycobacterial or other bacterial organism, a fungus or a virus;
(b) infection by a parasite;
(c) prevention of rejection of a graft.
(d) augmenting a vaccination response

2. Use according to claim 1, wherein the medicament further comprises an anti-infective agent, an oncolytic agent, an interferon, an interleukin, tumor necrosis factor, a transforming growth factor, leukemia inhibitory factor, a colony stimulating factor, or an anesthetic.

3. Use according to claim 3, wherein the medicament is for treatment of Mycobacterium tuberculosis and further comprises an anti-infective agent selected from isoniazid, rifampin, streptomycin, and at least one of the antibiotics of the pyrazinamide group.

4. Use according to claim 2, wherein the mycobacterial organism is Mycobacterium leprae.

5. Use according to claim 4, wherein the medicament further comprises dapsone, rifampin, or clofazimine.

6. Use according to claim 2, wherein the medicament is for the treatment of a fungal infection which is candidiasis, aspergillosis, blastomycosis, chromoblastomycosis, coccidiomycosis, cryptococcosis, histoplasmosis, mucormycosis, paracoccidiodomycosis, pseudallescheriasis, or sporotichosis.

7. Use according to claim 6, wherein the medicament further comprises amphotericin B, flucytosine, ketoconazole, fluconazole, or itraconazole.

8. Use according to any preceding claim, wherein the infection is by a parasite which is of a leishmania species or a plasmodium species.

9. Use according to any preceding claim, wherein the medicament which is manufactured provides a dose of about 1 to 10 μg/kg of the hosts' body weight.

10. Use according to any preceding claim, wherein the medicament is for treatment of humans, avians, bovines, equines, procines or fish.

11. Use according to any preceding claim, wherein the medicament is for treatment of the kidney, bone, lung, skin, stomach, small intestine or colon.

12. Use according to any preceding claim, wherein the medicament is for the treatment of Dengue virus, influenza virus, a hepatitis virus, or a herpesvirus.

13. Use according to any preceding claim, wherein X is leucine or isoleucine.

14. A pharmaceutical composition comprising a therapeutically effective amount of a dipeptide having the formula X-Tryptophan or a pharmaceutically acceptable salt thereof, wherein X is leucine or isoleucine; and a pharmaceutically acceptable carrier.

15. A composition of claim 14, wherein the dipeptide is present in the composition at a concentration of about 10 to 200 μg/ml.

16. A composition of claim 14 or 15, further comprising an anti-infective agent, an oncolytic agent, an interferon, are interleukin, tumor necrosis factor, a transforming growth factor, leukemia inhibitory factor, a colony stimulating factor, or an anesthetic.

17. A method for treating a host organism suffering from any of the indications set out in claims 1, 4, 6, 8 or 12 which comprises administering to the host a therapeutically effective amount of a dipeptide-containing medicament whose manufacture is referred to in any of claims 1 to 13 or a pharmaceutical composition as claimed in any of claims 14 to 16.


SEQUENCE LISTING (2 sequences)

SEQ ID NO: 1 - protein, 4 aa, Artificial Sequence
  MOD_RES (1)..(4): X= any amino acid
XWXW

SEQ ID NO: 2 - protein, 6 aa, Artificial Sequence
  MOD_RES (1)..(6): X=any amino acid
XWXWXW